(19) United States
(12) Patent Application Publication
(10) Pub. No.: US 2023/0268039 A1
(43) Pub. Date: Aug. 24, 2023

(54) METHODS, SYSTEMS, AND COMPUTER-READABLE MEDIA FOR DECREASING PATIENT PROCESSING TIME IN A CLINICAL SETTING

(71) Applicant: Januity Inc., Bellevue, WA (US)

(72) Inventors: Dinh Tran, MD, Renton, WA (US); Ajainder Shergill, DO, Danville, CA (US); Bob Thong, MD, Seattle, WA (US)

(21) Appl. No.: 18/140,383

(22) Filed: Apr. 27, 2023

Related U.S. Application Data
(63) Continuation-in-part of application No. 17/339,669, filed on Jun. 4, 2021.

(51) Int. Cl. G16H 10/60; G16H 50/30; G16H 50/20; G16H 40/67; G10L 25/66; G06V 40/16

(52) U.S. Cl. CPC G16H 10/60; G16H 50/30; G16H 50/20; G16H 40/67; G10L 25/66; G06V 40/174

(57) ABSTRACT

Techniques may include methods and systems for decreasing patient processing time in a clinical setting. An identity of patient may be identified based on identification (ID) information associated with the patient. The system may communicate with an Electronic Medical Record (EMR) platform to access medical data associated with the patient. Speech data associated with the patient may be acquired via a speech recording component. The speech data associated with the patient may be provided to a speech analysis component. The speech analysis component may generate first analytics based at least in part on the speech data associated with the patient. The system may communicate with the EMR platform to update the medical data associated with the patient based at least in part on the speech data associated with the patient.

### CROSS REFERENCE TO RELATED APPLICATION

This patent application is a continuation-in-part of U.S. patent application Ser. No. 17/339,669, filed Jun. 4, 2021, which is hereby incorporated by reference, in its entirety.

### BACKGROUND

Currently, a patient's journey through medical clinics' workflow is fragmented and time-consuming. Usually, an appointment time slot for a clinic visit is 20 to 30 minutes. However, a great part of the time may be taken up by administrative tasks such as check-in, rooming, retrieving medical records of the patient, and the like. As such, there is little time left for actual patient care and meaningful interaction between patients and physicians. Not surprisingly, the quality of patient care and outcomes may suffer from inefficiencies of the clinic workflow.

### BRIEF DESCRIPTION OF THE DRAWINGS

The Detailed Description is set forth with reference to the accompanying figures. The use of the same reference numbers in different figures indicates similar or identical items. Furthermore, the drawings may be considered as providing an approximate depiction of the relative sizes of the individual components within individual figures. However, the drawings are not to scale, and the relative sizes of the individual components, both within individual figures and between the different figures, may vary from what is depicted. In particular, some of the figures may depict components as a certain size or shape, while other figures may depict the same components on a larger scale or differently shaped for the sake of clarity.

FIG. 1 illustrates an example apparatus for decreasing patient processing time in a clinical setting according to implementations of this disclosure.

FIG. 2 illustrates an example environment where a terminal device associated with a patient, an apparatus in a clinical setting, an Electronic Medical Record (EMR) platform, and a terminal device associated with a care provider interoperate to decrease patient processing time in the clinical setting according to implementations of this disclosure.

FIG. 3A, FIG. 3B, FIG. 3C, FIG. 3D, FIG. 3E, and FIG. 3F are schematic flow diagrams illustrating an example process for decreasing patient processing time in the clinical setting according to implementations of this disclosure.

FIG. 4 illustrates an example page that may be presented on an interface of an apparatus in a clinical setting according to implementations of this disclosure.

FIG. 5A, FIG. 5B, FIG. 5C, FIG. 5D, FIG. 5E, FIG. 5F, and FIG. 5G illustrate a table of example use cases for decreasing patient processing time in a clinical setting according to implementations of this disclosure.

FIG. 6 illustrates an embodiment of a schematic depiction of a system of an apparatus and the componentry associated with the apparatus that performs the features according to this disclosure.

FIG. 7A, FIG. 7B, FIG. 7C, and FIG. 7D illustrate flow diagrams illustrating an example process for decreasing patient processing time in the clinical setting according to implementations of this disclosure.

FIG. 8 illustrates examples of physiological traits associated with a patient that may be useful for generating predictive analytics associated with the patient.

### DETAILED DESCRIPTION

This disclosure is generally directed to methods, systems, and computer-readable media for providing medical service. More particularly, this disclosure is directed to methods, systems, and computer-readable media for decreasing patient processing time in a clinical setting.

In various implementations, the techniques described herein may decrease patient processing time in a clinical setting. An identity of a patient may be verified based on identification (ID) information associated with the patient. An apparatus may communicate with an EMR platform to access previously entered medical data associated with the patient, when the patient is a returning patient; or create a new account associated with the patient, when the patient is a new patient. Instructions for acquiring instantaneous physiological data associated with the patient may be presented via the interface of the apparatus. The instantaneous physiological data associated with the patient may be acquired via one or more physiological data acquiring components of the apparatus. The apparatus may communicate with the EMR platform to update the medical data associated with the patient on the EMR platform based on the instantaneous physiological data associated with the patient and provide one or more recommendations for further care based on the medical data associated with the patient.

Throughout this disclosure, the EMR platform may include, but is not limited to, electronic medical record platforms, electronic health record platforms, other patient condition record platforms, and the like. An electronic medical record, or EM-R, may be the digital version of patient medical records that a doctor or pharmacy keeps on hand to keep track of medical records electronically from appointment to appointment.

Throughout this disclosure, the physiological data associated with the patient may refer to vitals parameters such as blood pressure, heart rate, pulse oxygen level, body temperature, respiratory rate, and the like, body parameters such as weight, height, body mass index (BMI), and the like. Biometric identifiers may refer to the distinctive, measurable characteristics used to label and describe individuals. The rooming process may refer to the process of gathering and inputting the patient's medical information, such as vitals, the reason for the visit, current medication list, past medical/social history, etc. Point-of-care testing (POC) may refer to medical diagnostic testing at or near the point of care—that is, at the time and place of patient care. Progress Notes may refer to medical records where care providers record a patient's clinical status or achievements such as patient's symptoms, exam findings, an overall diagnostic impression, plans for patient's care, and the like. The chief complaint may refer to a concise statement describing the symptom, problem, condition, diagnosis, physician-recommended return, or other reason for a medical encounter. The term “medication reconciliation” may refer to the process of identifying the most accurate list of all medications that the patient is taking, including drug name, dosage, frequency, and route, by comparing the medical data obtained from the EMR and the patient.

The techniques described herein may acquire and arrange the medical data and the instantaneous physiological data associated with the patient in an efficient way, decreasing the patient processing time in a clinical setting, facilitating the interaction between patients and medical care providers, streamlining the clinics' workflow, and/or improving the quality of care delivery.

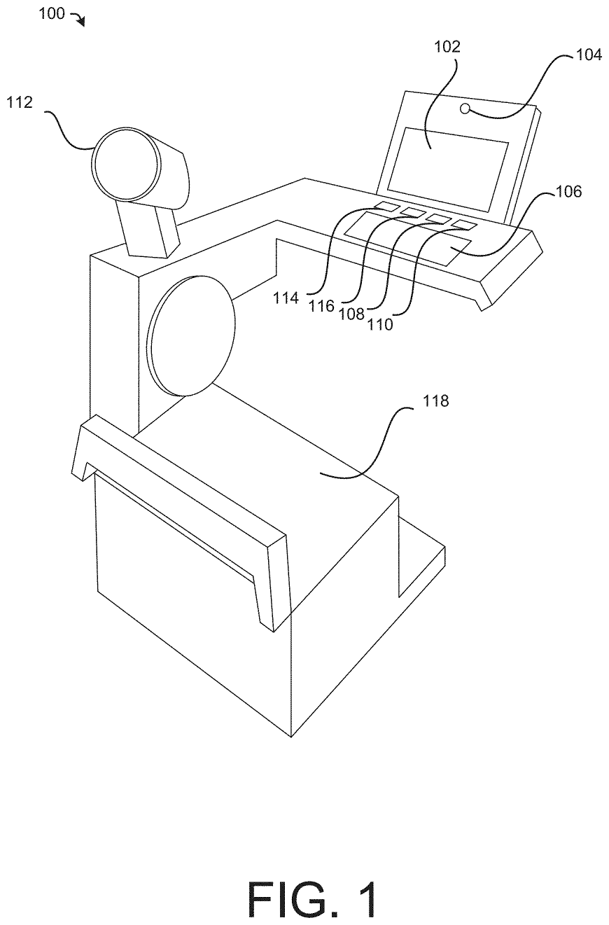

FIG. 1 illustrates an example apparatus 100 for decreasing patient processing time in a clinical setting according to implementations of this disclosure. In implementation, the apparatus 100 may include, but is not limited to, medical kiosks, check-in kiosks, interactive kiosks, self-service kiosks, smart kiosks, diagnostic kiosks, test kiosks, etc. In implementation, the apparatus 100 may be used in a clinical setting for the patient to conduct a self-check-in/rooming process.

Referring to FIG. 1, the apparatus 100 may include one or more processors (not shown), memory (not shown), an interface 102, a camera system 104, an input component 106, a payment component 108, a biometric identifier acquiring component 110, and one or more physiological data acquiring components.

The memory may be coupled to the one or more processors and may store thereon computer-readable instructions executable by the one or more processors.

The interface 102 may be coupled to the one or more processors and is configured to present information to the patient and/or receive information entered by the patient.

The camera system 104 may be coupled to the one or more processors, and is configured to capture images, for example, of objects and/or persons. In implementation, the camera system 104 may include, but is not limited to, optical cameras, digital cameras, webcams, infrared cameras, etc. The camera system 104 may be further configured to perform zooming, panning, and/or tilting when taking the picture. For example, the camera system may be configured to take pictures of skin areas of the patient for a care provider such as a dermatologist to review.

The input component 106 may be coupled to the one or more processors and is configured to allow input of data and/or information to the apparatus 100 for processing. In implementation, the input component 106 may include, but is not limited to, keyboards, mice, touchpads, handwriting pads, scanners, voice input devices, etc.

The payment component 108, may be coupled to the one or more processors, and is configured to process payment. In implementation, the payment component 108 may include, but is not limited to, a chip reader, a card swipe slot, a contactless payment device, a Point of Sale (POS) terminal, a payment code scanner, etc.

The biometric identifier acquiring component 110 may be coupled to the one or more processors and is configured to acquire a biometric identifier associated with the patient. In implementation, the biometric identifier may include, but is not limited to, fingerprint, palm veins, face recognition, Deoxyribonucleic Acid (DNA), palm print, hand geometry, iris recognition, retina, odor, typing rhythm, gait, keystroke, signature, voice, etc.

The one or more physiological data acquiring components may be coupled to the one or more processors and are configured to acquire physiological data associated with the patient. The one or more physiological data acquiring components may include, but are not limited to, a blood pressure cuff 112, a body temperature sensor 114, a pulse oximeter 116, and a weight scale 118.

The blood pressure cuff 112 is configured to acquire the blood pressure (including at least systolic blood pressure and diastolic blood pressure) associated with the patient.

The body temperature sensor 114 is configured to acquire the body temperature associated with the patient. In implementation, the body temperature sensor 114 may include, but is not limited to, an oral thermometer, an ear (tympanic) thermometer, a forehead (temporal) thermometer, a digital thermometer, a mercury (liquid in glass) thermometer, an infrared thermometer, etc.

The pulse oximeter 116 is configured to acquire the blood oxygen saturation level associated with the patient.

The weight scale 118 is configured to acquire the weight associated with the patient.

Additionally, and/or alternatively, the apparatus 100 may further include one or more point-of-care testing components (not shown). In implementation, the one or more point-of-care testing components may include, but is/are not limited to, an ultrasound scan component, a urine dipstick test component, an electrocardiogram (EKG) sensor, a rapid strep test component, a flu test component, a glucose check component, an Hemoglobin A1c (HbA1c) check component, a thyroid-stimulating hormone (TSH) test component, a lipid test component, a coronavirus disease (COVID-19) test component, an edema test component, an ultrasound exam component, an eye exam component, etc.

The apparatus 100 is configured to perform operations and processes described throughout this disclosure. Details of the operations and processes are not repeated here.

Additionally, the apparatus 100 may be further configured to run algorithms and/or Artificial Intelligence (AI) mechanisms. The algorithms and/or AI mechanisms may be configured to process and/or arrange the medical data associated with the patient and the instantaneous physiological data associated with the patient.

The algorithms and/or AI mechanisms may be configured to provide the one or more recommendations for the care provider to review, confirm/approve, deny/sign off, or correct/edit. Additional details of the one or more recommendations for the care are described throughout this disclosure and are not repeated here.

The algorithms and/or AI mechanisms may be configured to automatically transform the medical data associated with the patient into an enhanced format. For example, the algorithms and/or AI mechanisms may be configured to arrange information such as the reason for the visit, the medical condition, the allergy information, the medication list, and the physiological data associated with the patient in a coherent format and flow. For example, the algorithms and/or AI mechanisms may be configured to grammatically check and/or correct the input text (pronoun, complete sentences, etc.) For example, when the patient enters the description of symptoms and/or complaints, the algorithms and/or AI mechanisms may be configured to provide standard templates corresponding to the symptoms and/or complaints. For example, the patient may enter “back pain” as a complaint, and the algorithms and/or AI mechanisms may provide “point tenderness to palpation of lumbar at midline, limited range of motion (ROM)” corresponding to the complaint.

The algorithms and/or AI mechanisms may be configured to keep the Progress Notes for the care provider to review. The algorithms and/or AI mechanisms may be configured to provide intelligent organization/trending of medical data over a select time interval (i.e., diabetes control, weight, etc.) for the care provider to review to expedite the medical decision making.

The algorithms and/or AI mechanisms may be configured to perform telehealth operations to expand deployment to other healthcare settings, such as nursing homes, for remote monitoring, diagnosing, and providing healthcare.

Additionally, the algorithms and/or AI mechanisms may be assessed and finetuned continuously based on data and feedback from practice.

The apparatus 100 described herein may acquire and arrange the medical data associated with the patient in an efficient way, facilitating the interaction between patients and medical care providers, decreasing the patient processing time in a clinical setting, streamlining the clinics' workflow, and/or improving the quality of care delivery.

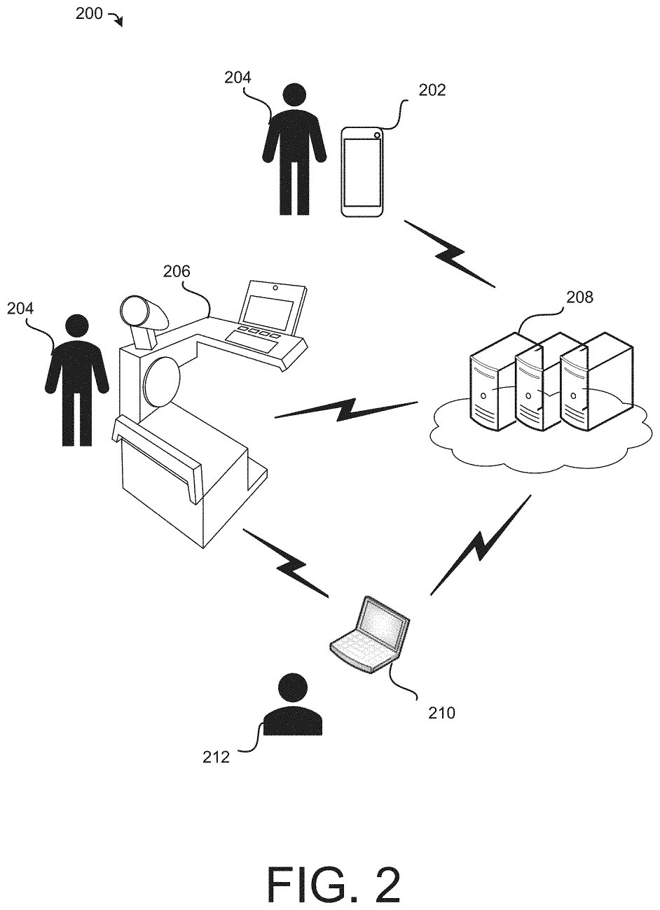

FIG. 2 illustrates an example environment 200 where a terminal device 202 associated with a patient 204, an apparatus 206 in a clinical setting, an EMR platform 208, and a terminal device 210 associated with a care provider 212 interoperate to decrease patient processing time in the clinical setting according to implementations of this disclosure. In some instances, the apparatus 206 in the clinical setting and the terminal device 210 associated with the care provider 212 may be taken as a system.

Referring to FIG. 2, the patient 204 may create a new account and/or review/verify/input medical data associated with the patient 204, for example, on an application (App) or a web portal through the terminal device before going for the clinic visit at home or other places. In implementation, the App or the web portal may be a software that is responsible for a variety of functions, including but are not limited to, sending auto-reminders, providing recommendations for further care, keeping progress notes, etc. Additionally, and/or alternatively, the software may be integrated with the EMR platform via a third party.

For example, the patient 204 may be a new patient 204, and there may be no medical data associated with the patient 204 on the EMR platform. In that case, the patient 204 may create an account and enter the medical data associated with the patient 204 via the App or the web portal. The medical data associated with the patient 204 may include, but is not limited to, the insurance information associated with the new patient 204, the demographic information associated with the new patient 204, the payment method, the medication list associated with the new patient 204, the medical/social history information associated with the new patient 204, etc. The medical/social history information may include, but is not limited to, tobacco use, alcohol use, drug use, etc. Alternatively, the patient 204 may do nothing here, and the patient 204 may be given the option to create an account and enter the medical data associated with the patient 204 at the clinic.

For example, the patient 204 may be an existing/returning patient, and there may be previously entered medical data associated with the patient 204 on the EMR platform. In that case, the patient 204 may log in to the account associated with the patient 204 to review/verify/edit the medical data associated with the patient 204. Alternatively, the patient 204 may do nothing here, and the patient 204 may be given the option to review/verify/edit the medical data associated with the patient 204 at the clinic.

Additionally, the patient 204 may sign/eSign documents and/or agreements via the App or the web portal, for example, Health Insurance Portability and Accountability Act (HIPAA) policy, payment responsibility/bill insurance authorization document, etc. Additionally, and/or alternatively, the patient 204 may do nothing here, and the patient 204 may be given the option to sign/eSign the documents and/or agreements at the clinic.

Additionally, information regarding the reason for the visit/chief complaint associated with the patient 204 may be gathered via the App or the web portal. The reason for the visit/chief complaint may include, but is not limited to, new complaints, medication refills, follow-ups, Annual Physical Exam/Well Child Visits/AWE, etc. Additionally, and/or alternatively, the existing/returning patient may do nothing here, and the existing/returning patient may be given the option to enter the reason for the visit/chief complaint at the clinic.

In implementation, the App or the web portal may gather the reason for the visit/chief complaint associated with the patient 204 in templates. The templates may include, but are not limited to, a new complaint template, a medication refill template, a follow-up template, an Annual Physical Exam/Well Child Visits/AWE template, etc.

For example, the new complaint template may include entries of the duration of symptoms, the severity of symptoms, the symptom onset, what makes the symptoms better, what makes the symptoms worse, etc. Examples of new complaints may include, but are not limited to, pain, discomfort, upper and lower respiratory infections, cold symptoms, depression, anxiety, any acute illnesses, etc. Additionally, and/or alternatively, the new complaint template may include entries for free text regarding additional complaints/concerns.

For example, the medication refill template may include entries regarding how is the patient 204 doing on the medication, whether there are any side effects, whether the patient 204 takes the medication daily as instructed, whether the condition improved/controlled while the patient 204 is on medication, etc. Additionally, and/or alternatively, the medication refill template may include entries for free text regarding the additional thoughts/concerns.

For example, the follow-up template may include entries regarding any changes since most recent visit, how is the patient 204 doing over the interval (doing better or worse), whether the treatment/therapy (if any) helps, etc. Additionally, and/or alternatively, a follow-up template may include entries for free text regarding additional thoughts/concerns.

For example, the Annual Physical Exam/Well-Child Visits/AWE template may include instructions such as “Physical, Complete Checkup”, and the like.

In implementation, the templates are configurable and upgradable. The templates may be implemented via algorithms and/or AI mechanisms. Additionally, the templates may be improved to include smart and specific questions for common medical issues.

Additionally, the App or the web portal may perform medication reconciliation based on the medical data associated with the patient 204 to obtain a reconciled medication list associated with the patient 204. Additionally, and/or alternatively, the medication reconciliation may be done by the apparatus 206 at the clinic.

Additionally, the terminal device 202 may run an App to allow the terminal device 202 to capture a set of physiological data associated with the patient 204. For example, the App may allow the terminal device 202 to scan the face of the patient 204 to capture a stress level associated with the patient 204.

Additionally, the terminal device 202 may auto-sync the medical data associated with the patient 204 from home medical devices such as a home blood pressure monitoring device and a home blood glucose monitoring device, for example, via Bluetooth connections.

The terminal device 202 may communicate with the EMR platform 208 to create the account, save and/or update the medical data associated with the patient 204.

The patient 204 may go for the clinic visit. The front desk staff at the clinic may recommend that the patient 204 check in via the apparatus 206.

The apparatus 206 may verify the identity of the patient 204 based on ID information associated with the patient 204. The ID information may include, but is not limited to, biometric identifiers, Email ID, etc. In implementation, the biometric identifier may include, but is not limited to, fingerprint, palm veins, face recognition, DNA, palm print, hand geometry, iris recognition, retina, odor, typing rhythm, gait, keystroke, signature, voice, etc.

The apparatus 206 may request, via the interface of the apparatus, ID information associated with the patient 204. The apparatus 206 may receive the ID information associated with the patient 204. The apparatus 206 may determine, based on the ID information associated with the patient 204, the identity associated with the patient 204 to correlate the identity with the medical data associated with the patient 204.

The apparatus 206 may communicate with the EMR platform to access previously entered medical data associated with the patient 204, when the patient 204 is a returning patient; or create a new account associated with the patient 204, when the patient 204 is a new patient 204.

For example, the patient 204 is a new patient 204, and there may be no medical data associated with the patient 204 on the EMR platform. In that case, the patient 204 may create an account and enter the medical data associated with the patient 204 via the apparatus 206. The medical data associated with the patient 204 may include, but is not limited to, the insurance information associated with the new patient 204, the demographic information associated with the new patient 204, the payment method, the medication list associated with the new patient 204, the medical/social history information associated with the new patient 204, etc. The medical/social history information may include, but is not limited to, tobacco use, alcohol use, drug use, etc. Alternatively, the patient 204 may have created the account before coming to the visit and may do nothing here.

For example, the patient 204 may be an existing/returning patient, and there may be previously entered medical data associated with the patient 204 on the EMR platform. In that case, the patient 204 may log in to the account associated with the patient 204 to review/verify/edit the medical data associated with the patient 204. Alternatively, the patient 204 may have reviewed/verified/edited the medical data associated with the patient 204 before coming to the visit and may do nothing here.

Additionally, the patient 204 may sign/eSign documents and/or agreements, for example, HIPAA policy, payment responsibility/bill insurance authorization document, etc. Alternatively, the patient 204 may have signed the documents and/or agreements and may do nothing here.

The apparatus 206 may present a request for a reason for a visit/chief complaint on the interface of the apparatus 206. In implementation, the reason for the visit/chief complaint may include, but is not limited to, new complaints, medication refills, follow-ups, Annual Physical Exam/Well Child Visits/Annual Wellness Exam (AWE), etc. In implementation, the apparatus may gather the reason for the visit/chief complaint associated with the patient 204 in templates. Additional details of the templates are described throughout this disclosure and are not repeated here.

The apparatus 206 may receive the reason for the visit/chief complaint associated with the patient 204 via the interface of the apparatus. In implementation, the apparatus 206 may automatically provide a standard template corresponding to the reason for the visit/chief complaint. For example, the patient 204 may enter “back pain” as a complaint, and apparatus 206 may generate a standard template “point tenderness to palpation of lumbar at midline, limited range of motion (ROM)” corresponding to the complaint.

The apparatus 206 may communicate with the EMR platform to update the medical data associated with the patient 204 on the EMR platform based on the reason for the visit/chief complaint associated with the patient 204. Additionally, the standard template corresponding to the reason for the visit/chief complaint associated with the patient 204 may be automatically uploaded to the EMR platform.

The apparatus 206 may perform medication reconciliation based on the medical data associated with the patient 204 to obtain a reconciled medication list associated with the patient 204. Additionally, and/or alternatively, the medication reconciliation may also be done, for example, via the terminal device associated with the patient 204 from home prior to the clinic visit. Additionally, if the patient 204 requests medication refills, the apparatus 206 may automatically provide the medication refill request for the care provider to approve or disapprove.

The apparatus 206 may present, via the interface of the apparatus, instructions for acquiring instantaneous physiological data associated with the patient 204. For example, the apparatus 206 may present via the interface of the apparatus 206, instructions for acquiring the temperature associated with the patient 204, the oxygen level associated with the patient 204, the weight associated with the patient 204, the blood pressure associated with the patient 204, and the like. Additional details are described throughout this disclosure and are not repeated here.

The apparatus 206 may acquire the instantaneous physiological data associated with the patient 204 via the one or more physiological data acquiring components of the apparatus 206.

In implementation, the instantaneous physiological data may include, but is not limited to, systolic blood pressure, diastolic blood pressure, body temperature, weight, heart rate, pulse rate, respiratory rate, pulse oximetry, etc.

In implementation, the one or more physiological data acquiring components may include, but are not limited to, a blood pressure cuff, a body temperature sensor, a pulse oximeter, and weight scale, etc.

The patient 204 may follow the instructions for acquiring instantaneous physiological data to use one or more physiological data acquiring components of the apparatus 206 to acquire the instantaneous physiological data associated with the patient 204.

For example, the patient 204 may use the body temperature sensor to check the body temperature associated with the patient 204. The patient 204 may put his/her figure on the pulse oximeter to check the oxygen level associated with the patient 204. The patient 204 may step on the weight scale to check the weight associated with the patient 204. The patient 204 may put his/her arm in the blood pressure cuff to check the blood pressure associated with the patient 204.

Additionally, and/or alternatively, the apparatus 206 may further include one or more point-of-care testing components. In implementation, the one or more point-of-care testing components may include, but is/are not limited to, an ultrasound scan component, a urine dipstick test component, an EKG sensor, a rapid strep test component, a flu test component, a glucose check component, an HbA1c check component, a TSH test component, a lipid test component, a COVID-19 test component, an edema (leg swelling) test component, an ultrasound exam component, an eye exam component, etc. In implementation, the edema test component may be a laser sensor. Additionally, and/or alternatively, the camera system 104 may be a point-of-care testing component, that captures rashes or any abnormal skin findings to be interactive with a remote dermatologist for diagnostic feedback.

The apparatus 206 may communicate with the EMR platform to update the medical data associated with the patient 204 on the EMR platform based on the instantaneous physiological data associated with the patient 204 and provide one or more recommendations for further care based on the medical data associated with the patient 204. For example, the one or more recommendations for further care may be based on the age and/or the medical conditions associated with the patient.

In implementation, the one or more recommendations for further care include, but is/are not limited to, 1) providing advice and/or recommendations about a medical condition, 2) diagnosing and/or treating a medical condition, 3) providing referral services for a medical condition, 4) prescribing medicine for a medical condition, 5) periodically monitoring a medical condition, 6) providing follow-up checks for a medical condition, 7) providing routine check-up services, 8) providing advice, counseling, and/or recommendations about medical and/or health matters, 9) providing a course of treatment for a medical condition, 8) providing health counseling, 10) providing health information, 12) providing wellness counseling, and/or 13) providing wellness information. As may be appreciated, other or additional recommendations may be provided. In implementation, the one or more recommendations may include an option of doing nothing.

Additionally, the apparatus 206 may convert the medical data associated with the patient 204 and/or the instantaneous physiological data associated with the patient 204 into a graph over a time interval and automatically upload the graph to the EMR platform.

The apparatus 206 may notify the care provider that the patient is ready for the visit. For example, the apparatus 206 may set up an EMR alert to be sent to the terminal device 210 to notify the care provider 212 that the patient 204 is ready for the visit.

The terminal device 210 associated with the care provider 212 may present the reason for the visit/chief complaint associated with the patient 204, the medical data associated with the patient 204, the instantaneous physiological data associated with the patient 204, and the one or more recommendations for further care that are pending. Additionally, and/or alternatively, the terminal device 210 associated with the care provider 212 may present the medical data associated with the patient 204 and/or the instantaneous physiological data associated with the patient 204 over a time interval in a graphic format.

The care provider 212 may review the reason for the visit/chief complaint associated with the patient 204, the medical data associated with the patient 204, the instantaneous physiological data associated with the patient 204, and/or the one or more recommendations for further care that are pending.

The patient 204 and the care provider 212 may meet in a room and conduct the clinic visit. Additionally, and/or alternatively, the patient 204 and the care provider 212 may conduct the clinic visit virtually/remotely.

The care provider 212 may confirm/approve, deny/sign off, or correct/edit the one or more recommendations for further care via the terminal device 210. The terminal device 210 associated with the care provider 212 may receive a confirmation, a denial, and/or a correction of a respective recommendation of the one or more recommendations entered by the care provider 212. Additionally, and/or alternatively, the care provider 212 may enter diagnostic information associated with the patient 204.

The terminal device 210 associated with the care provider 212 may communicate with the EMR platform to update the medical data associated with the patient 204 based on the confirmation, the denial, and/or the correction of the respective recommendation of the one or more recommendations for further care, and/or diagnostic information associated with the patient 204.

With the environment 200, the terminal device 202 associated with the patient 204, the apparatus 206 in the clinical setting, the EMR platform 208, and the terminal device 210 associated with the care provider 212 interoperate to acquire and arrange the medical data and the instantaneous physiological data associated with the patient in an efficient way, decreasing the patient processing time in a clinical setting, facilitating the interaction between patients and medical care providers, streamlining the clinics' workflow, and/or improving the quality of care delivery.

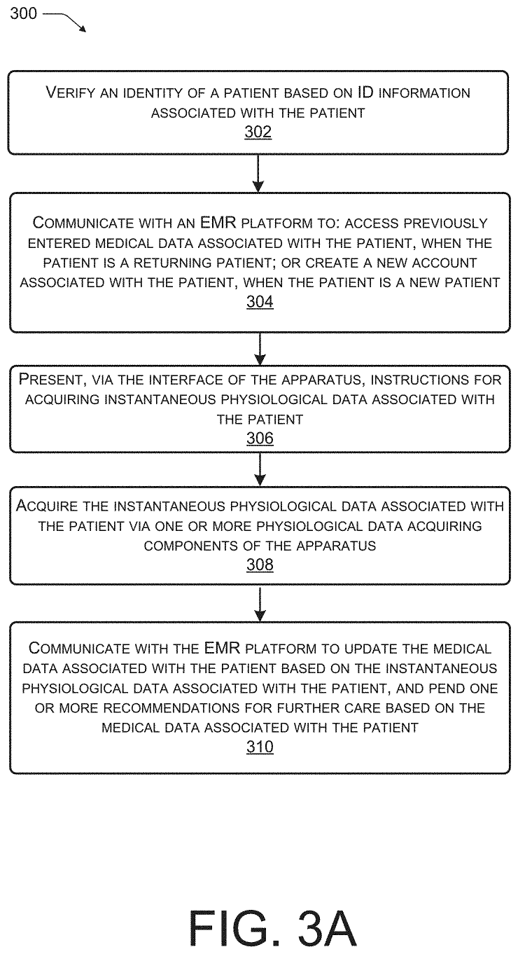

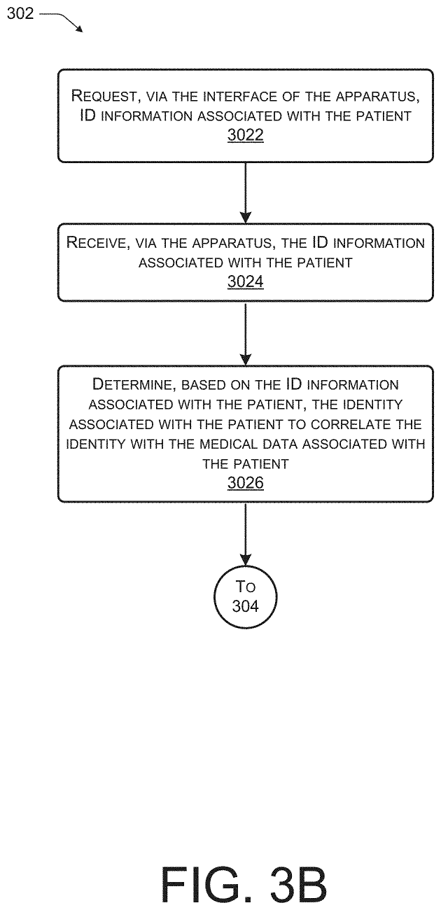

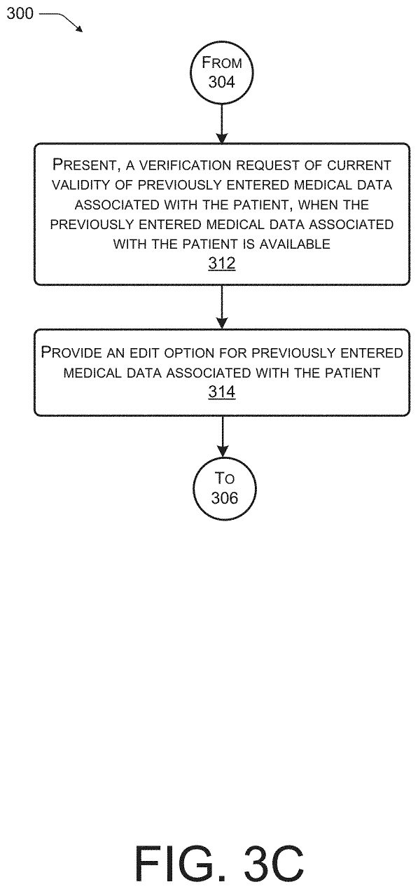

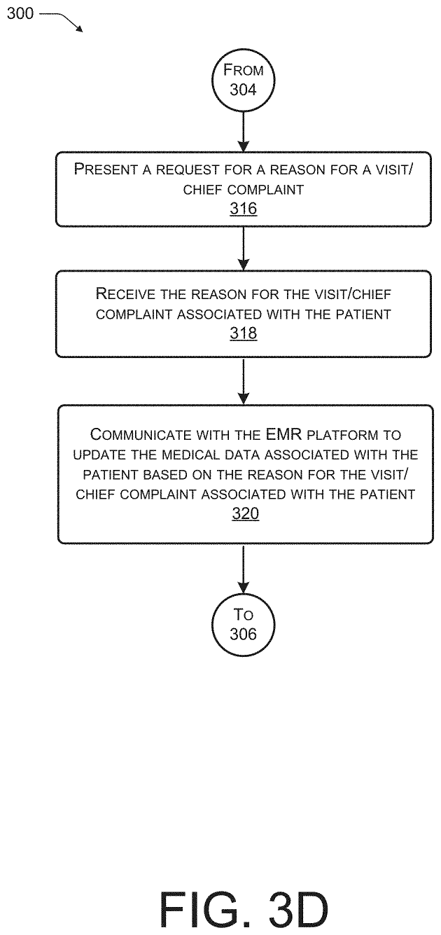

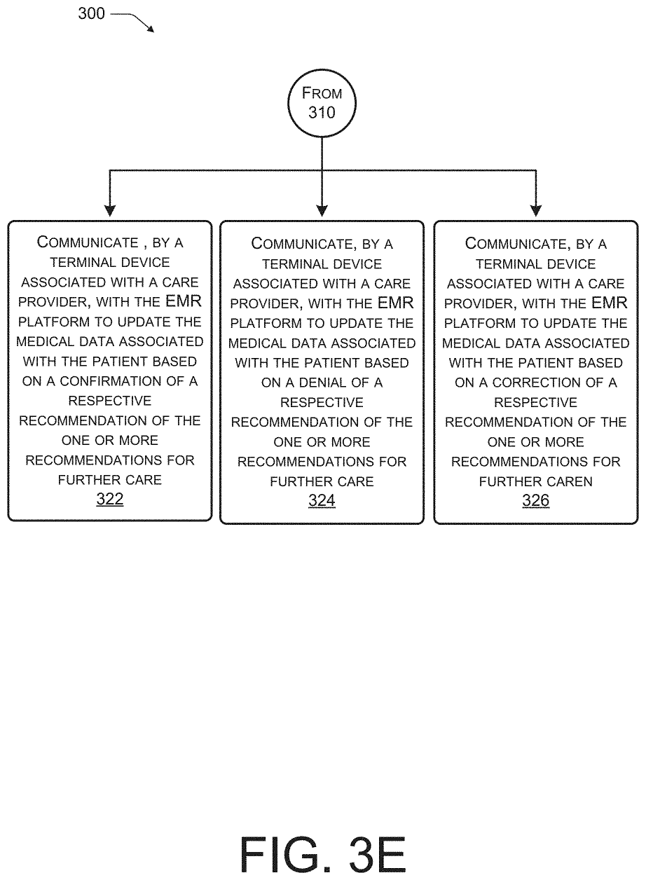

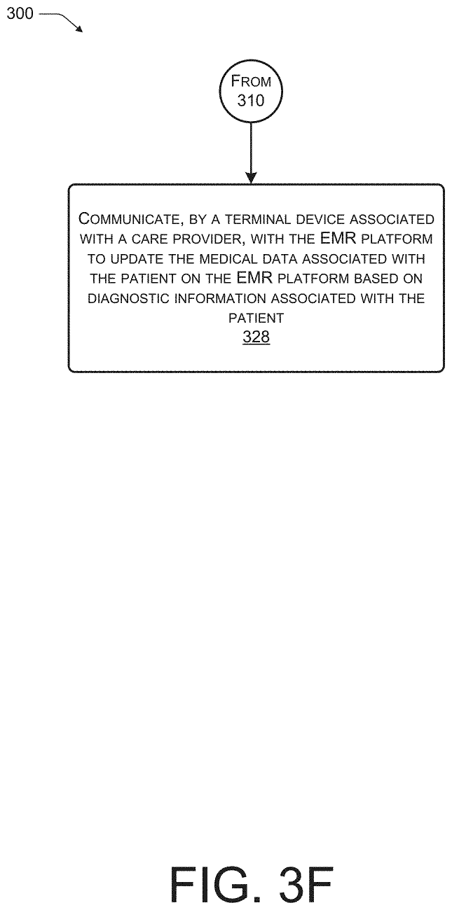

FIG. 3A, FIG. 3B, FIG. 3C, FIG. 3D, FIG. 3E, and FIG. 3F are schematic flow diagrams illustrating an example process 300 for decreasing patient processing time in the clinical setting according to implementations of this disclosure.

Referring to FIG. 3A, the process 300 may include:

At 302, operations may include verifying an identity of a patient based on ID information associated with the patient.

At 304, operations may include communicating with an EMR platform to: access previously entered medical data associated with the patient, when the patient is a returning patient; or create a new account associated with the patient, when the patient is a new patient.

At 306, operations may include presenting, via the interface of the apparatus, instructions for acquiring instantaneous physiological data associated with the patient. In implementation, the instantaneous physiological data may include, but is not limited to, systolic blood pressure, diastolic blood pressure, body temperature, weight, heart rate, pulse rate, respiratory rate, or pulse oximetry.

For example, the apparatus may present, via the interface of the apparatus, instructions for acquiring the temperature associated with the patient, the oxygen level associated with the patient, the weight associated with the patient, the blood pressure associated with the patient, and the like.

At 308, operations may include acquiring the instantaneous physiological data associated with the patient via one or more physiological data acquiring components of the apparatus.

In implementation, the one or more physiological data acquiring components may include, but are not limited to, a blood pressure cuff, a body temperature sensor, a pulse oximeter, or a weight scale.

For example, the patient may use the body temperature sensor to check the body temperature associated with the patient. The patient may put his/her figure on the pulse oximeter to check the oxygen level associated with the patient. The patient may step on the weight scale to check the weight associated with the patient. The patient may put his/her arm in the blood pressure cuff to check the blood pressure associated with the patient.

Additionally, and/or alternatively, the medical data associated with the patient and/or the instantaneous physiological data associated with the patient may be converted into a graph over a time interval.

Additionally, and/or alternatively, the apparatus may further include one or more point-of-care testing components (not shown). In implementation, the one or more point-of-care testing components may include, but are not limited to, an ultrasound scan component, a urine dipstick test component, an EKG sensor, a rapid strep test component, a flu test component, a glucose check component, an HbA1c check component, a TSH test component, a lipid test component, a COVID-19 test component, an edema test component, an ultrasound exam component, an eye exam component, etc.

At 310, operations may include communicating with the EMR platform to update the medical data associated with the patient based on the instantaneous physiological data associated with the patient and provide one or more recommendations for further care based on the medical data associated with the patient. For example, the one or more recommendations for further care may be based on the age and/or the medical conditions associated with the patient.

In implementation, the one or more recommendations for further care include, but is/are not limited to, 1) providing advice and/or recommendations about a medical condition, 2) diagnosing and/or treating a medical condition, 3) providing referral services for a medical condition, 4) prescribing medicine for a medical condition, 5) periodically monitoring a medical condition, 6) providing follow-up checks for a medical condition, 7) providing routine check-up services, 8) providing advice, counseling, and/or recommendations about medical and/or health matters, 9) providing a course of treatment for a medical condition, 8) providing health counseling, 10) providing health information, 12) providing wellness counseling, and/or 13) providing wellness information. As may be appreciated, other or additional recommendations may be provided. In implementation, the one or more recommendations may include a single option of doing nothing. In implementation, the care provider may be able to confirm/approve, deny/sign off, and/or correct/edit the recommendations.

Additional details of the one or more recommendations for the care are described throughout this disclosure and are not repeated here.

Referring to FIG. 3B, 302 may further include:

At 3022, operations may include requesting, via the interface of the apparatus, ID information associated with the patient. The ID information may include, but is not limited to, biometric identifiers, Email ID, etc.

At 3024, operations may include receiving, via the apparatus, the ID information associated with the patient. For example, the patient may input a biometric identifier via a biometric identifier acquiring component of the apparatus. Additionally, and/or alternatively, the patient may enter an Email ID via the interface of the apparatus.

At 3026, operations may include determining, based on the ID information associated with the patient, the identity associated with the patient to correlate the identity with the medical data associated with the patient.

Referring to FIG. 3C, between 304 and 306, the process 300 may further include:

At 312, operations may include presenting a verification request of current validity of previously entered medical data associated with the patient, when the previously entered medical data associated with the patient is available.

For example, for example, the patient may review/verify the insurance information associated with the patient, the demographic information associated with the patient, the payment method, the medication list associated with the patient, the medical/social history information associated with the patient, etc. For example, the medical/social history information may include, but is not limited to, tobacco use, alcohol use, drug use, etc.

At 314, operations may include providing an edit option for previously entered medical data associated with the patient.

Referring to FIG. 3D, between 304 and 306, the process 300 may further include:

At 316, operations may include presenting a request for a reason for a visit/chief complaint.

In implementation, the reason for the visit/chief complaint may include, but is not limited to, new complaints, medication refills, follow-ups, Annual Physical Exam/Well Child Visits/Annual Wellness Exam (AWE), etc. In implementation, the apparatus may gather the reason for the visit/chief complaint associated with the patient in templates. The templates may include, but are not limited to, a new complaint template, a medication refill template, a follow-up template, an Annual Physical Exam/Well Child Visits/AWE template, etc.

For example, the new complaint template may include entries of the duration of symptoms, the severity of symptoms, the symptom onset, what makes the symptoms better, what makes the symptoms worse, etc. Examples of new complaints may include, but are not limited to, pain, discomfort, upper and lower respiratory infections, cold symptoms, depression, anxiety, any acute illnesses, etc. Additionally, and/or alternatively, the new complaint template may include entries for free text regarding additional complaints/concerns.

For example, the medication refill template may include entries regarding how the patient is doing on the medication, whether there are any side effects, whether the patient takes the medication daily as instructed, whether the condition improved/controlled while the patient is on medication, etc. Additionally, and/or alternatively, the medication refill template may include entries for free text regarding the additional thoughts/concerns.

For example, the follow-up template may include entries regarding any changes since most recent visit, how the patient is doing over the interval (doing better or worse), whether the treatment/therapy (if any) helps, etc. Additionally, and/or alternatively, a follow-up template may include entries for free text regarding additional thoughts/concerns.

For example, the Annual Physical Exam/Well-Child Visits/AWE template may include instructions such as “Physical, Complete Checkup”, and the like.

In implementation, the templates are configurable and upgradable. The templates may be implemented via algorithms and/or AI mechanisms. Additionally, the templates may be improved to include smart and specific questions for common medical issues.

At 318, operations may include receiving the reason for the visit/chief complaint associated with the patient. In implementation, a standard template corresponding to the reason for the visit associated with the patient may be automatically provided. For example, the patient may enter “back pain” as a complaint, and the apparatus may generate a standard template “point tenderness to palpation of lumbar at midline, limited range of motion (ROM)” corresponding to the complaint.

At 320, operations may include communicating with the EMR platform to update the medical data associated with the patient based on the reason for the visit/chief complaint associated with the patient. Additionally, the standard template corresponding to the reason for the visit associated with the patient may be automatically uploaded to the EMR platform.

Referring to FIG. 3E, the process 300 may further include:

At 322, operations may include communicating, by a terminal device associated with a care provider, with the EMR platform to update the medical data associated with the patient based on a confirmation of a respective recommendation of the one or more recommendations for further care.

At 324, operations may include communicating, by a terminal device associated with a care provider, with the EMR platform to update the medical data associated with the patient based on a denial of a respective recommendation of the one or more recommendations for further care.

At 326, operations may include communicating with the EMR platform to update the medical data associated with the patient on the EMR platform based on the correction of the respective recommendation.

Referring to FIG. 3F, the process 300 may further include:

At 328, operations may include communicating, by a terminal device associated with a care provider, with the EMR platform to update the medical data associated with the patient on the EMR platform based on diagnostic information associated with the patient.

With the process 300, the medical data and the instantaneous physiological data associated with the patient may be acquired and arranged in an efficient way, decreasing the patient processing time in a clinical setting, facilitating the interaction between patients and medical care providers, streamlining the clinics' workflow, and/or improving the quality of care delivery.

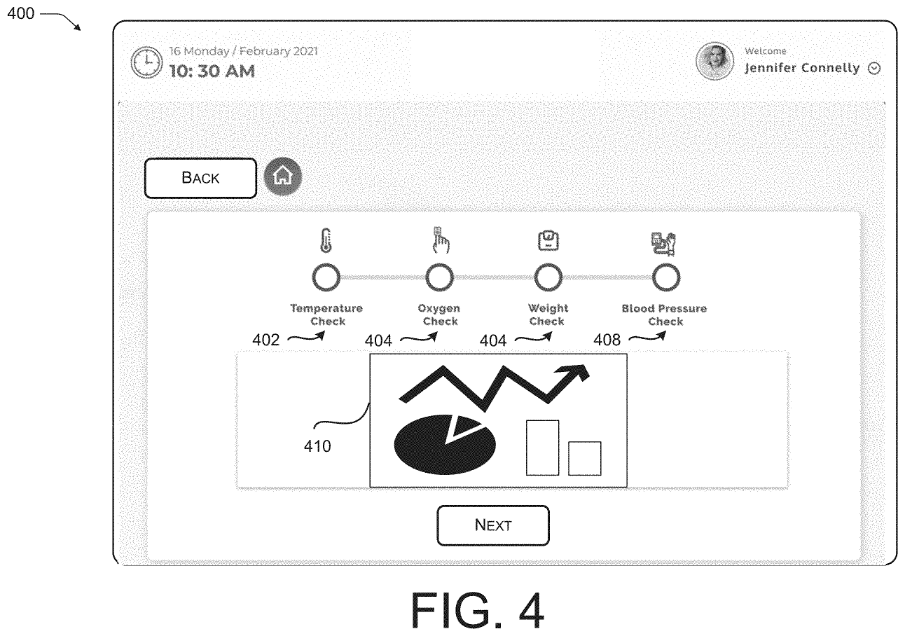

FIG. 4 illustrates an example page 400 which may be presented on an interface of an apparatus in a clinical setting according to implementations of this disclosure. Referring to FIG. 4, the page 400 may show instructions for acquiring instantaneous physiological data associated with the patient, for example, “temperature check” 402, “oxygen check” 404, “weight check” 406, and “blood pressure check” 408. The page 400 may include a graph area 410.

In implementation, the patient may read and follow the instructions to use one or more physiological data acquiring components of the apparatus in the clinical setting to acquire the instantaneous physiological data associated with the patient. The one or more physiological data acquiring components may include, but are not limited to, a blood pressure cuff, a body temperature sensor, a pulse oximeter, and a weight scale.

For example, the patient may use the body temperature sensor to check the body temperature associated with the patient. The patient may put his/her figure on the pulse oximeter to check the oxygen level associated with the patient. The patient may step on the weight scale to check the weight associated with the patient. The patient may put his/her arm in the blood pressure cuff to check the blood pressure associated with the patient. The apparatus in the clinical setting may convert the acquired instantaneous physiological data into a graphic format and present the graph in the graph area 410.

The graph area 410 may be configured to show the medical data associated with the patient and/or the instantaneous physiological data associated with the patient in a graph over a time interval.

In implementation, after the check-in is completed, the patient may wait in the waiting area to be called by medical assistants (MA). During the waiting time, the apparatus in the clinical setting may automatically communicate with the EMR platform to save/update the medical data associated with the patient based on the gathered information and provide any medication refill requests for the care provider to approve of and/or sign off. Accordingly, the care provider may see the patient on time with more designated face-to-face time with the patient.

The techniques described herein may acquire and arrange the medical data associated with the patient in an efficient way, facilitating the interaction between patients and medical care providers, decreasing the patient processing time in a clinical setting, streamlining the clinics' workflow, and/or improving the quality of care delivery.

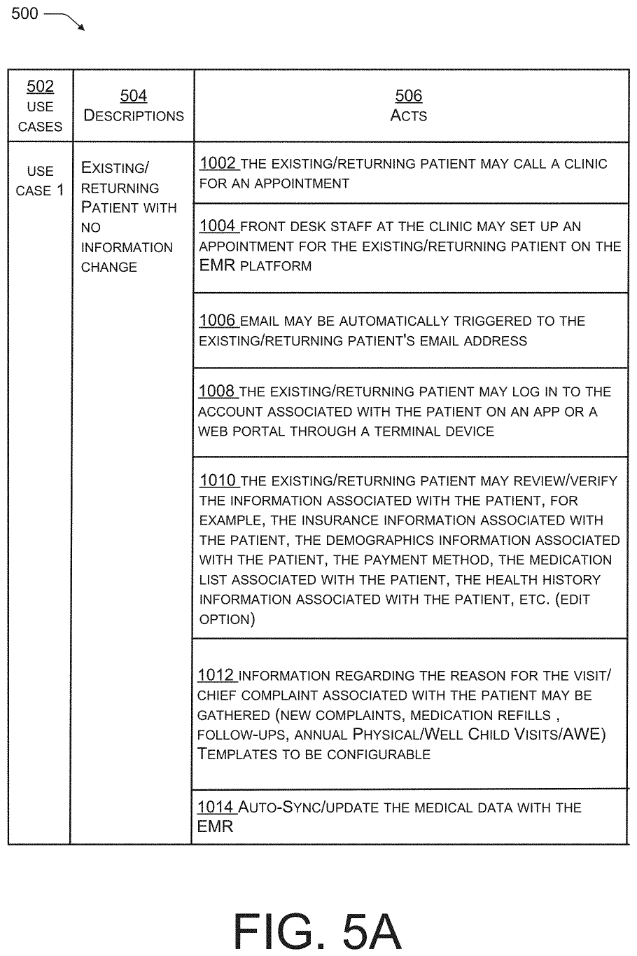

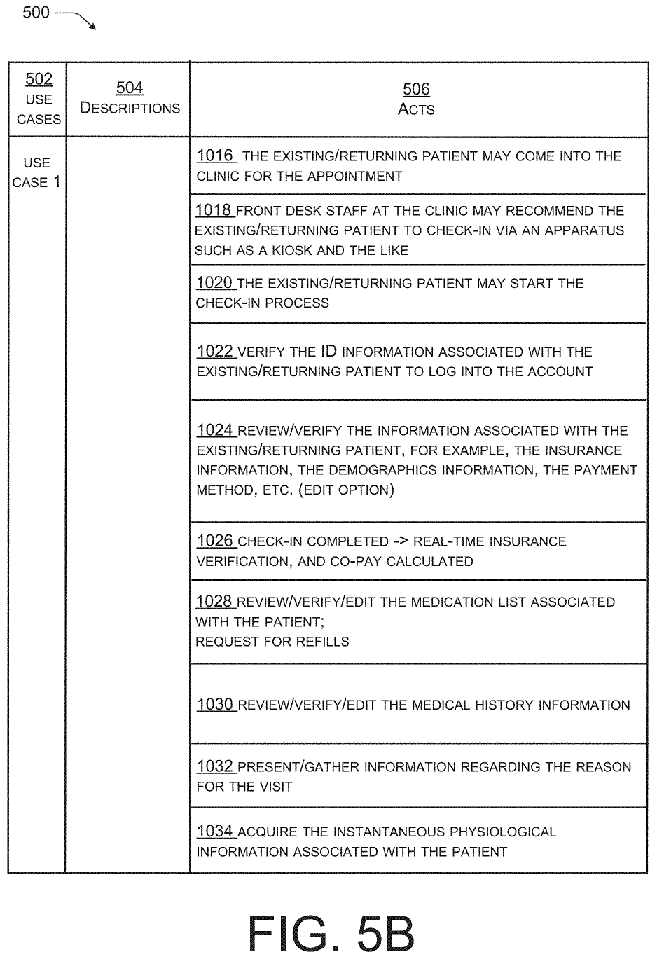

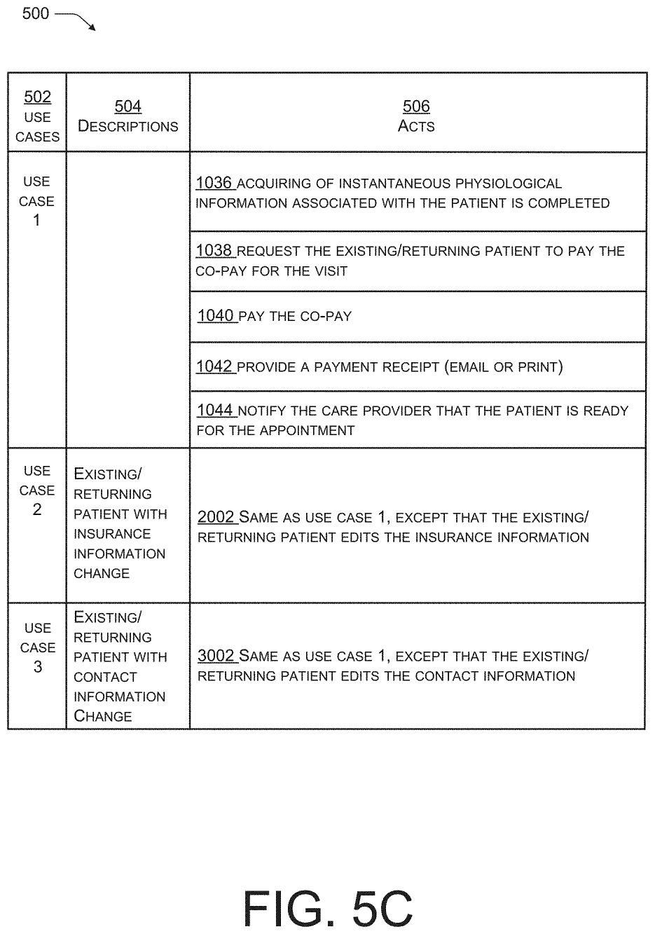

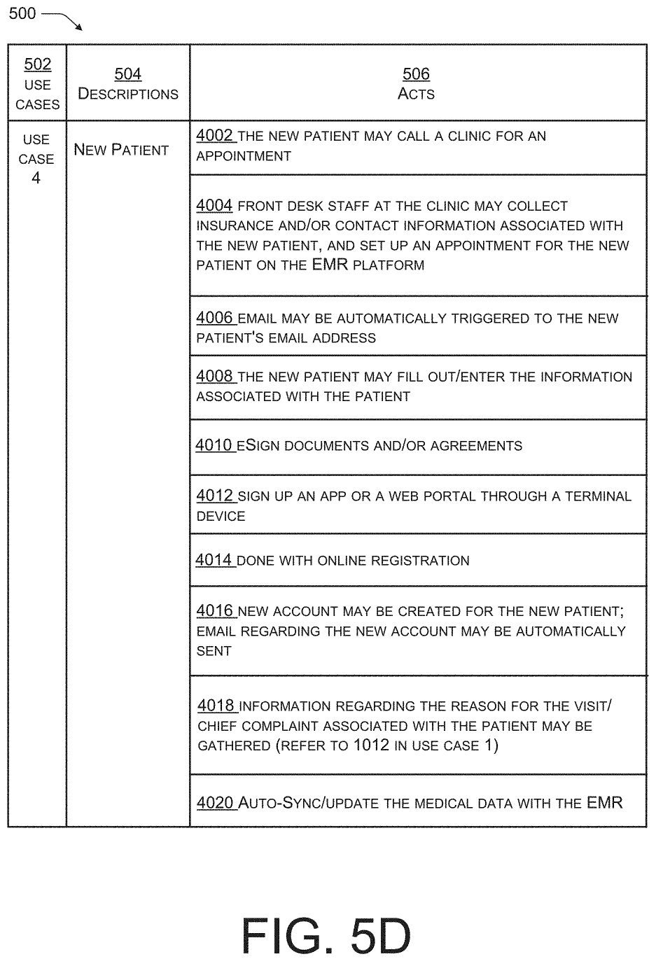

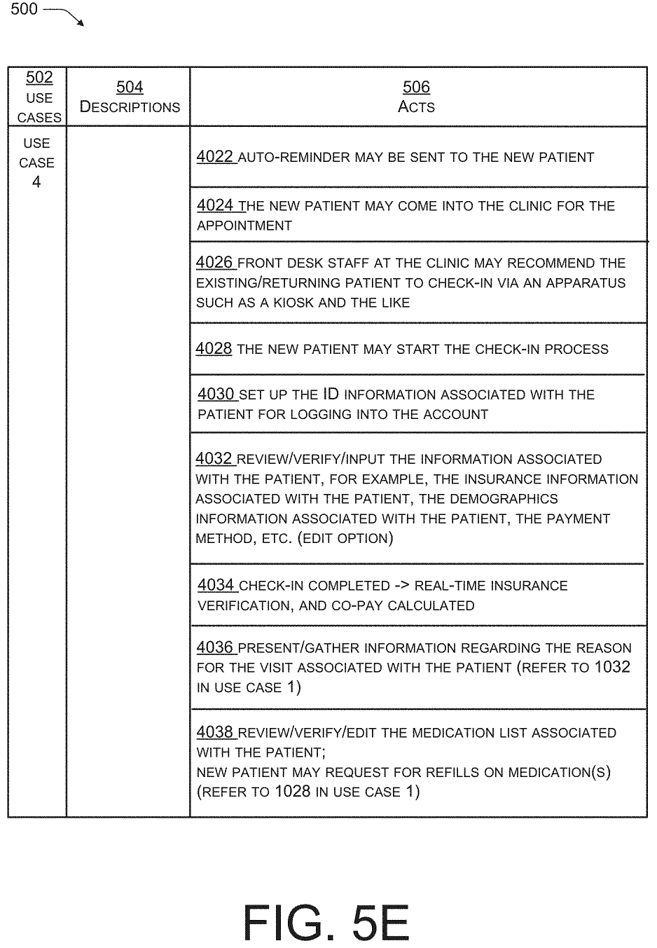

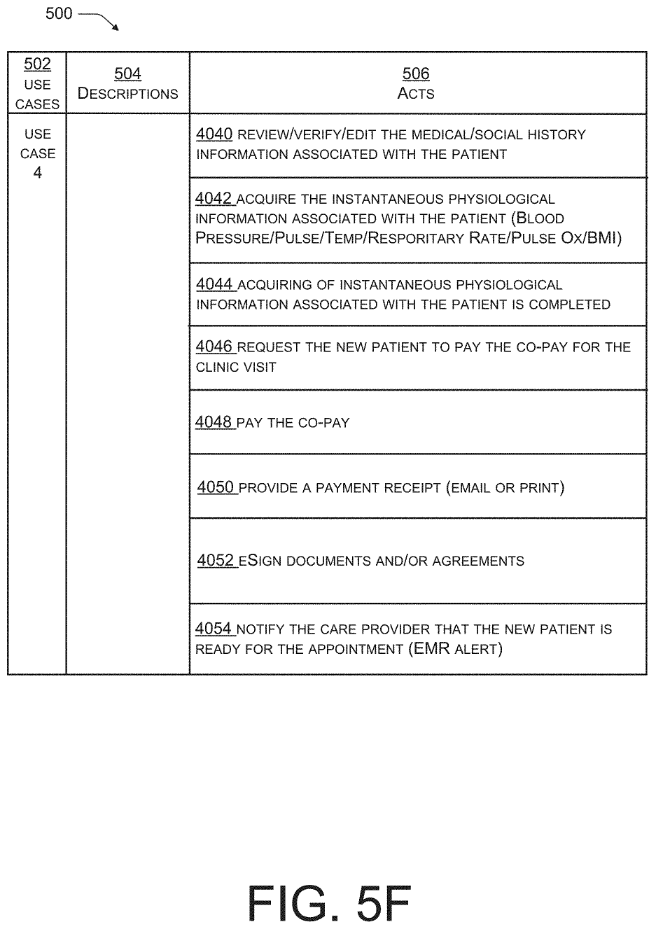

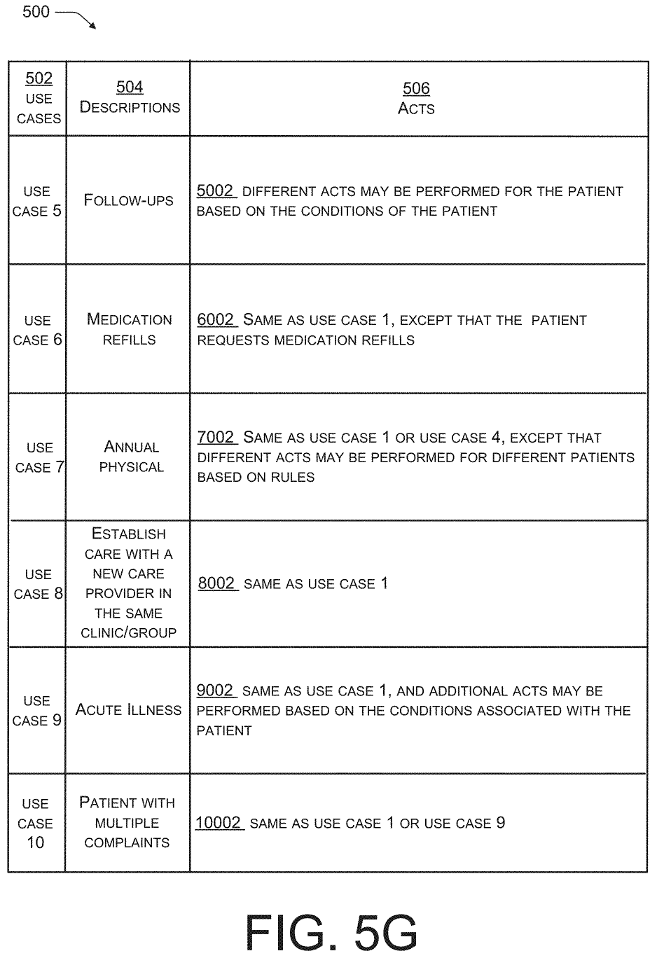

FIG. 5A, FIG. 5B, FIG. 5C, FIG. 5D, FIG. 5E, FIG. 5F, and FIG. 5G illustrate a table 500 of example use cases for decreasing patient processing time in a clinical setting according to implementations of this disclosure.

Referring to FIG. 5A, column 502 shows names of use cases. Column 504 shows descriptions of the use cases. Column 506 shows acts that may be performed for the use cases.

Use case 1 may be a case where an existing/returning patient with no information change makes a clinic visit.

At 1002, the existing/returning patient may call a clinic for an appointment.

At 1004, the front desk staff at the clinic may set up an appointment for the existing/returning patient on the EMR platform.

At 1006, an email may be automatically triggered to the existing/returning patient's email address regarding the appointment. The email may request the patient to review/verify/edit the information associated with the existing/returning patient, for example, the insurance information associated with the existing/returning patient, the demographic information associated with the existing/returning patient, the payment method, the medication list associated with the existing/returning patient, the medical/social history information associated with the existing/returning patient, etc. For example, the medical/social history information may include, but is not limited to, tobacco use, alcohol use, drug use, etc.

At 1008, the existing/returning patient may log in to the account associated with the existing/returning patient on an app or a web portal through a terminal device such as a computer, a mobile phone, and the like, to review/verify/edit the information associated with the existing/returning patient.

At 1010, the existing/returning patient may review/verify the information associated with the existing/returning patient, for example, the insurance information associated with the existing/returning patient, the demographic information associated with the existing/returning patient, the payment method, the medication list associated with the existing/returning patient, the medical/social history information associated with the existing/returning patient, etc. For example, the medical/social history information may include, but is not limited to, tobacco use, alcohol use, drug use, etc. Additionally, and/or alternatively, the existing/returning patient may be given options to edit the information associated with the existing/returning patient.

At 1012, information regarding the reason for the visit/chief complaint associated with the existing/returning patient may be gathered. The reasons for the visit may include, but are not limited to, new complaints, medication refills, follow-ups, Annual Physical Exam/Well Child Visits/Annual Wellness Exam (AWE), etc. In implementation, the App or the web portal may gather the reason for the visit/chief complaint associated with the existing/returning patient in templates. The templates may include, but are not limited to, a new complaint template, a medication refill template, a follow-up template, an Annual Physical Exam/Well Child Visits/AWE template, etc.

For example, the new complaint template may include entries of the duration of symptoms, the severity of symptoms, the symptom onset, what makes the symptoms better, what makes the symptoms worse, etc. Examples of new complaints may include, but are not limited to, pain, discomfort, upper and lower respiratory infections, cold symptoms, depression, anxiety, any acute illnesses, etc. Additionally, and/or alternatively, the new complaint template may include entries for free text regarding additional complaints/concerns.

For example, the medication refill template may include entries regarding how the patient is doing on the medication, whether there are any side effects, whether the patient takes the medication daily as instructed, whether the condition improved/controlled while the patient is on medication, etc. Additionally, and/or alternatively, the medication refill template may include entries for free text regarding the additional thoughts/concerns.

For example, the follow-up template may include entries regarding any changes since most recent visit, how the patient is doing over the interval (doing better or worse), whether the treatment/therapy (if any) helps, etc. Additionally, and/or alternatively, a follow-up template may include entries for free text regarding additional thoughts/concerns.

For example, the Annual Physical Exam/Well-Child Visits/AWE template may include instructions such as “Physical, Complete Checkup”, and the like.

In implementation, the templates are configurable and upgradable. The templates may be implemented via algorithms and/or AI mechanisms. Additionally, the templates may be improved to include smart and specific questions for common medical issues.

Additionally, and/or alternatively, the existing/returning patient may do nothing here, and the existing/returning patient may be given the option to enter the reason for the visit/chief complaint at the clinic.

At 1014, the terminal device may auto-sync/update the medical data associated with the existing/returning patient on the EMR via the App or the web portal.

In implementation, acts described with reference to FIG. 5A may be performed when the patient is at home or other places before going for the clinic visit. Additionally, and/or alternatively, the patient may do nothing before going for the clinic visit. The patient may be given the option to enter and/or update the medical data associated with the existing/returning patient at the clinic.

Referring to FIG. 5B, at 1016, the existing/returning patient may come into the clinic for the appointment.

At 1018, the front desk staff at the clinic may recommend that the existing/returning patient check in via an apparatus such as a kiosk and the like.

At 1020, the existing/returning patient may start the check-in process via the apparatus.

At 1022, the existing/returning patient may verify the ID information associated with the existing/returning patient to log into the account associated with the existing/returning patient via the apparatus. In implementation, the ID information may include, but is not limited to, biometric identifiers, Email ID, etc. For example, the existing/returning patient may log into the account associated with the existing/returning patient using a biometric identifier, an Email ID, and the like. In implementation, the biometric identifier may include, but is not limited to, fingerprint, palm veins, face recognition, DNA, palm print, hand geometry, iris recognition, retina, odor, typing rhythm, gait, keystroke, signature, voice, etc.

At 1024, the existing/returning patient may review/verify the information associated with the existing/returning patient, for example, the insurance information associated with the existing/returning patient, the demographic information associated with the existing/returning patient, the payment method, etc. Additionally, and/or alternatively, the existing/returning patient may have options to edit the information associated with the existing/returning patient.

At 1026, the check-in process may be completed. The completion of the check-in process may trigger a real-time insurance verification, and the co-pay for the visit may be calculated.

At 1028, the existing/returning patient may review/verify/edit the medication list associated with the existing/returning patient. For example, the existing/returning patient may add/delete medications from the medication list, and/or edit the dosage of the medication, the frequency of the medication, and the like. However, the existing/returning patient may not be able to edit any controlled substances in the medication list.

Additionally, the existing/returning patient may request refills on medication(s). For example, the existing/returning patient may request refills on medication(s) by clicking the check boxes. The medication refill requests may be automatically pended for the care provider such as a physician to approve of and/or sign off on the care provider's end.

At 1030, the existing/returning patient may review/verify/edit the medical/social history information associated with the existing/returning patient. For example, the existing/returning patient may add new diagnoses to the medical/social history information associated with the existing/returning patient. Additionally, there may be some diagnoses in the medical/social history that are not editable, for example, Attention Deficit Disorder (ADD), deficit hyperactivity disorder (ADHD), and the like. Additionally, the existing/returning patient may not be able to delete pre-existing diagnoses.

At 1032, the apparatus may present/gather information regarding the reason for the visit associated with the existing/returning patient. The reasons for the visit may include, but are not limited to, new complaints, medication refills, follow-ups, Annual Physical Exam/Well Child Visits/AWE, etc. For example, the existing/returning patient may have already entered the reason for the visit prior to the clinic visit, then the existing/returning patient may do nothing at this stage. Additionally, and/or alternatively, the existing/returning patient may edit the reason for the visit associated with the existing/returning patient via the apparatus. Additionally, and/or alternatively, the existing/returning patient may have not entered the reason for the visit prior to the clinic visit, then the existing/returning patient may enter the reason for the visit via the apparatus.

In implementation, the apparatus may be configured to regulate/process the information gathered regarding the reason for the visit associated with the existing/returning patient. For example, the apparatus may be configured to designate a maximum of four lines for additional data entry of a brief summary of symptoms/the reason for the visit. The apparatus may be configured to auto-populate the chief complaint information into the History of Present Illness (HPI) section of Progress Note for the care provider such as the physician to review. The auto-populating feature may be customizable. For example, the auto-populating feature may be turned on/off, depending on the care provider's choice. The apparatus may be configured to process the information gathered regarding the reason for the visit associated with the existing/returning patient. For example, the apparatus may be configured to replace the pronoun “I” with the term “the patient” in the text entered.

At 1034, the apparatus may acquire the instantaneous physiological data associated with the existing/returning patient. In implementation, the instantaneous physiological data may include, but is not limited to, systolic blood pressure, diastolic blood pressure, body temperature, weight, heart rate, pulse rate, respiratory rate, or pulse oximetry. Additionally, the apparatus may calculate/derive other physiological data associated with the existing/returning patient such as BMI and the like.

Referring to FIG. 5C, at 1036, the acquiring of instantaneous physiological data associated with the existing/returning patient is completed.

At 1038, the apparatus may request the existing/returning patient to pay the co-pay for the clinic visit. Additionally, color codes for payment may be made to show different status of the payment. For example, the co-pay shown in red means that the co-pay has not been paid, and the co-pay shown in blue means that the co-pay is paid.

At 1040, the existing/returning patient may pay the co-pay via a payment component of the apparatus. For example, the existing/returning patient may have selected/confirmed the payment method previously, and the existing/returning patient may use the payment method on file to pay the co-pay. Additionally, and/or alternatively, the existing/returning patient may change/edit the payment method to pay the co-pay, for example, by adding an existing/returning card.

At 1042, the apparatus may provide a payment receipt to the existing/returning patient. For example, the apparatus may email the payment receipt to the email address associated with the existing/returning patient. Additionally, and/or alternatively, the apparatus may print the payment receipt. Additionally, and/or alternatively, the existing/returning patient may choose no receipt.

At 1044, the apparatus may notify the care provider that the existing/returning patient is ready for the appointment. For example, the apparatus may notify the care provider that the existing/returning patient is ready for the appointment via an EMR alert.

Use case 2 may be a case where an existing/returning patient with insurance information change makes a clinic visit.

At 2002, acts for use case 2 may be the same as the acts described with reference to use case 1 except for acts described with reference to 1010 and/or 1024. For use case 2, the existing/returning patient may edit the insurance information associated with the existing/returning patient at 1010. Additionally, and/or alternatively, the existing/returning patient may edit the insurance information associated with the existing/returning patient at 1024.

Use case 3 may be a case where an existing/returning patient with contact information change makes a clinic visit.

At 3002, acts for use case 3 may be the same as the acts described with reference to use case 1 except for acts described with reference to 1010 and/or 1024. For use case 3, the existing/returning patient may edit the contact information associated with the existing/returning patient at 1010. Additionally, and/or alternatively, the existing/returning patient may edit the contact information associated with the existing/returning patient at 1024.

Referring to FIG. 5D, use case 4 may be a case where a new patient makes a clinic visit.

At 4002, the new patient may call a clinic for an appointment.

At 4004, the front desk staff at the clinic may collect insurance and/or contact information associated with the new patient and set up an appointment for the new patient on the EMR platform.

At 4006, an email may be automatically triggered to the new patient's email address regarding the appointment. The email may request the new patient to fill out/enter the information associated with the new patient, for example, the insurance information associated with the new patient, the demographic information associated with the new patient, the payment method, the medication list associated with the new patient, the medical/social history information associated with the new patient, etc. For example, the medical/social history information may include, but is not limited to, tobacco use, alcohol use, drug use, etc.

At 4008, the new patient may fill out/enter the information associated with the new patient, for example, the insurance information associated with the new patient, the demographic information associated with the new patient, the payment method, the medication list associated with the new patient, the medical/social history information associated with the new patient, etc. For example, the medical/social history information may include, but is not limited to, tobacco use, alcohol use, drug use, etc. Alternatively, the patient may do nothing here, and the patient may be given the option to enter and/or update the medical data associated with the patient at the clinic.

At 4010, the new patient may sign/eSign documents and/or agreements, for example, HIPAA policy, payment responsibility/bill insurance authorization document, etc. Alternatively, the new patient may do nothing here, and the new patient may be given the option to sign/eSign the documents and/or agreements at the clinic.

At 4012, the new patient may sign up for an app or a web portal through a terminal device such as a computer, a mobile phone, and the like. The app or a web portal may enable the patient to communicate with the care provider's office and/or access up-to-date medical data. The patient may be able to access appointments, lab results, vitals, and manage medications and other personal data via the app or the web portal.

At 4014, the new patient may complete the online registration on the app or the web portal.

At 4016, a new account associated with the new patient may be created via the app or the web portal.

Additionally, an email regarding the new account may be automatically sent to the email address of the new patient. In implementation, the email may include login information associated with the new patient for the new account. Additionally, the email may request the patient to enter information regarding the reason for the visit/chief complaint on the App or the web portal. Additionally, and/or alternatively, the reason for the visit/chief complaint may be gathered at the clinic.

At 4018, information regarding the reason for the visit/chief complaint associated with the new patient may be gathered. Acts for 4018 may be the same as acts described with reference to 1012 in use case 1. Details would not be repeated here.

At 4020, the terminal device may auto-sync/update the medical data associated with the new patient on the EMR via the App or the web portal.

Referring to FIG. 5E, at 4022, an auto-reminder may be sent to the patient, for example, from the EMR platform. The auto-reminder may remind the patient to review/update the information associated with the new patient such as the current medications list before the clinic visit. In implementation, the auto-reminder may be sent based on several rules. The rules may regulate conditions for sending the reminder such as the age range of the patient, how many days from the date of the most recent clinic visit, and so on. For example, rule 1 may be: sending the reminder for a patient whose age falls within a first range (such as 0 to 40), and whose date of the most recent clinic visit is more than a first number of days (such as 90 days). For example, rule 2 may be: sending the reminder for a patient whose age falls within a second range (such as 40 to 60), and whose date of the most recent clinic visit is more than a second number of days (such as 60 days). For example, rule 3 may be: sending the reminder for a patient whose age falls within a third range (such as more than 60), and whose date of the most recent clinic visit is more than a third number of days (such as 14 days). The rules described above are examples and this disclosure is not limited thereto. Additionally, the rules are configurable, for example, via an administrative user interface of the App or the web portal.

Additionally, the configurable features may apply to other use cases.

At 4024, the new patient may come into the clinic for the appointment.

At 4026, the front desk staff at the clinic may recommend the new patient to check-in via an apparatus such as a kiosk and the like.

At 4028, the new patient may start the check-in process via the apparatus.

At 4030, the new patient may set up the ID information associated with the new patient for logging into the account associated with the new patient via the apparatus. In implementation, the ID information may include, but is not limited to, biometric identifiers, Email ID, etc. The biometric identifier may include, but is not limited to, fingerprint, palm veins, face recognition, DNA, palm print, hand geometry, iris recognition, retina, odor, typing rhythm, gait, keystroke, signature, voice, etc.

At 4032, the new patient may review/verify/input the information associated with the new patient, for example, the insurance information associated with the new patient, the demographic information associated with the new patient, the payment method, etc. Additionally, and/or alternatively, the new patient may have options to edit the information associated with the new patient.

At 4034, the check-in process may be completed. The completion of the check-in process may trigger a real-time insurance verification, and the co-pay for the visit may be calculated.

At 4036, the apparatus may present/gather information regarding the reason for the visit associated with the new patient. Acts for 4036 here may be the same as acts described with reference to 1032 in use case 1. Details would not be repeated here.

At 4038, the new patient may review/verify/edit the medication list associated with the new patient. Additionally, the new patient may request refills on medication(s). Acts performed here may be the same as acts described with reference to 1028 in use case 1. Details would not be repeated here.

Referring to FIG. 5F, at 4040, the new patient may review/verify/edit the medical/social history information associated with the new patient. Acts performed here may be the same as acts described with reference to 1030 in use case 1. Details would not be repeated here.

At 4042, the apparatus may acquire the instantaneous physiological data associated with the new patient. Acts performed here may be the same as acts described with reference to 1034 in use case 1. Details would not be repeated here.

At 4044, the acquiring of instantaneous physiological data associated with the new patient is completed.

At 4046, the apparatus may request the new patient to pay the co-pay for the clinic visit.

At 4048, the new patient may pay the co-pay via a payment component of the apparatus. For example, the new patient may have selected/confirmed the payment method previously, and the new patient may use the payment method on file to pay the co-pay. Additionally, and/or alternatively, the new patient may change/edit the payment method to pay the co-pay, for example, by adding a new card.

At 4050, the apparatus may provide a payment receipt to the new patient. For example, the apparatus may email the payment receipt to the email address associated with the new patient. Additionally, and/or alternatively, the apparatus may print the payment receipt. Additionally, and/or alternatively, the new patient may choose no receipt.

At 4052, the new patient may eSign (digitally sign) documents and/or agreements, for example, HIPAA policy, payment responsibility/bill insurance authorization document, etc. Additionally, and/or alternatively, if the new patient has already signed/eSigned the documents and/or agreements at home or other places, the new patient may do nothing here.

At 4054, the apparatus may notify the care provider that the new patient is ready for the appointment. For example, the apparatus may set up an EMR alert to notify the care provider that the new patient is ready for the appointment.

Referring to FIG. 5G, use case 5 may be a case where a patient makes a clinic visit for follow-ups.

At 5002, different acts may be performed for the patient based on the conditions of the patient.

For example, for patients with hypertension/high blood pressure, the acts may include, but are not limited to, 1) pending a comprehensive metabolic panel (CMP) test, a lipid test, and an HbA1C test; 2) showing a graph of the blood pressure data associated with the patient acquired in the clinic over a configurable time interval (such as past 6 months); and 3) auto-syncing/updating the medical data associated with the patient on the EMR platform with any blood pressure data associated with the patient from any home blood pressure monitoring devices, for example, via Bluetooth connections.

Additionally, the graph of the blood pressure data associated with the patient may pop up when the care provider (such as a physician) reviews the medical data associated with the patient on the EMR platform, such that the care provider does not need to search for the graph. Additionally, and/or alternatively, the popping-up feature of the graph may be configured to be turned on/off.

For example, for patients with dyslipidemia/high cholesterol, the acts may include, but are not limited to, 1) pending a lipid test and an HbA1C test; and 2) showing a graph of the lipid test results associated with the patient over a configurable time interval.

Additionally, the graph of the lipid test results associated with the patient may pop up when the care provider (such as a physician) reviews the medical data associated with the patient on the EMR platform, such that the care provider does not need to search for the graph. Additionally, and/or alternatively, the popping-up feature of the graph may be configured to be turned on/off.

For example, for patients with a thyroid disorder, the acts may include, but are not limited to, 1) pending the TSH test with reflex T4; and 2) showing a graph of TSH results associated with the patient over a configurable time interval.

Additionally, the graph of TSH results associated with the patient may pop up when the care provider (such as a physician) reviews the medical data associated with the patient on the EMR platform, such that the care provider does not need to search for the graph. Additionally, and/or alternatively, the popping-up feature of the graph may be configured to be turned on/off.

For example, for patients with diabetes, the acts may include, but are not limited to, 1) pending an HbA1C test, a lipid test, a urine microalbumin/creatinine test, a basic metabolic panel (BMP) test, an optometry referral, and Pneumococcal Conjugate Vaccine (PCV13); and 2) showing a graph of the test results of HbA1c, urine microalbumin/creatinine, BMP or CMP associated with the patient over a configurable time interval (such as past one year); 3) auto-syncing/updating the medical data associated with the patient on the EMR platform with any home blood glucose data associated with the patient from any monitoring devices into the system, for example, via Bluetooth connections.

Additionally, the graph of the test results of HbA1c, urine microalbumin/creatinine, BMP or CMP associated with the patient may pop up when the care provider (such as a physician) reviews the medical data associated with the patient on the EMR platform, such that the care provider does not need to search for the graph. Additionally, and/or alternatively, the popping-up feature of the graph may be configured to be turned on/off.

For example, for patients with chronic obstructive pulmonary disease (COPD) or asthma, the acts may include, but are not limited to, 1) pending Pulmonary Function Tests (PFTs), Pulmonary Rehab, and PCV 13.

For example, for patients with congestive heart failure (CHF), the acts may include, but are not limited to, 1) pending a CMP test; 2) showing a graph of 2-Dimension Echocardiography (2D Echo) data associated with the patient over a configurable time interval; and 3) showing a graph of weights associated with the patient over a configurable time interval.

Additionally, the graph of the 2D Echo data and the graph of weights associated with the patient may pop up when the care provider (such as a physician) reviews the medical data associated with the patient on the EMR platform, such that the care provider does not need to search for the graphs. Additionally, and/or alternatively, the popping-up feature of the graphs may be configured to be turned on/off.

Use case 6 may be a case where a patient makes a clinic visit for medication refills.

At 6002, acts for use case 6 may be the same as the acts described with reference to use case 1 except for acts described with reference to 1012 and/or 1028. For use case 6, the patient may request medication refills at 1010 and/or 1028.

Use case 7 may be a case where a patient makes a clinic visit for an annual physical exam.

At 7002, acts for use case 7 may be the same as the acts described with reference to use case 1 or case 4 except that different acts may be performed for different patients based on rules related to, for example, the age range and other conditions of the patient.

For example, for patients who are women falling within a first age range (such as under 21), the acts may include, but are not limited to, pending Pap smear screening (for example, every 3 years) and chlamydia screening (for women 25 or less).

For example, for patients who fall within a second age range (such as over 45), the acts may include, but are not limited to, pending a lipid test, an HbA1C test, a CBC test, a CMP test, a TSH test with reflex T4, etc.

For example, for patients who are women falling within a third age range (such as over 30), the acts may include, but are not limited to, pending a Pap smear screening, a human papillomavirus (HPV), for example, every 5 years.

For example, for patients who fall within a fourth age range (such as over 50), the acts may include, but are not limited to, pending a colonoscopy exam, a lipid test, an A1C test, a CBC test, a CMP test, a TSH test with reflex T4, a mammogram screening (for women), etc.

For example, for patients who fall within a fifth age range (such as over 60), the acts may include, but are not limited to, pending a lipid test, an A1C test, a CMP test, a CBC test, a TSH test with reflex T4, shingles vaccination, colonoscopy referral, etc.

For example, for patients who fall within a sixth age range (such as over 65), the acts may include, but are not limited to, pending a lipid test, an A1C test, a CBC test, a CMP test, a TSH with reflex T4 test, a dual energy X-ray absorptiometry (DEXA) scan for women, Pneumovax 23 (PCV 23), etc.

Additionally, other rules may apply to determine the acts to be performed for the patient. For example, rule 1 may stipulate the age range and lab results. For example, rule 2 may stipulate the age range and screening tests (such as mammogram, colonoscopy, DEXA smay, and so on). For example, rule 3 may stipulate the age range, the time of the year, vaccination history, the recommendation of required vaccinations, and so on. For example, for September to February, the recommendation of Flu shot may be added.

The above rules are examples, and this disclosure is not limited thereto. The rules are configurable, upgradeable, and editable on the care provider's end.

Use case 8 may be a case where a patient makes a clinic visit to establish care with a new care provider in the same clinic/group.

At 8002, acts for use case 8 may be the same as the acts described with reference to use case 1.

Use case 9 may be a case where a patient makes a clinic visit due to acute illness, for example, a sore throat, knee/back pain, etc.

At 9002, acts for use case 9 may be the same as the acts described with reference to use case 1. Moreover, additional acts as follows may be performed based on the conditions associated with the patient.

For example, for patients with a sore throat, the acts may include, but are not limited to, 1) pending a rapid step test; and 2) pending order for amoxicillin 500 mg, 1 tab twice daily×10 days, 20 tabs, 0 refill.

For example, for patients with back pain, the acts may include, but are not limited to, 1) pending order for Flexeril, 10 mg qhs prn (before bedtime as needed), 30 tabs, 0 refills; and 2) pending a referral to physical therapy.

For example, for patients with knee pain, the acts may include, but are not limited to, 1) pending an X-ray exam; and 2) pending a referral to physical therapy.

For example, for patients with chest pain, the acts may include, but are not limited to, pending an EKG.

For example, for patients with Urinary Tract Infection (UTI)/urinary symptoms (such as dysuria, pain with urination, urinary frequency, urinary urgency, and the like), the acts may include, but are not limited to, 1) pending a urine dipstick test; 2) pending a urine culture test; 3) pending order for antibiotic (for example, Nitrofurantoin BID (twice a day)×5 days, 10 capsules, 0 refill). Additionally, and/or alternatively, the choice of antibiotic is configurable and may be based on prior urine culture sensitivities.

For example, for patients with flu-like symptoms (such as body aches, fatigue, fever, and the like), the acts may include, but are not limited to pending a flu test (for example, between September and May).

For example, for patients with ear pain, the acts may include, but are not limited to 1) pending order for Amoxicillin 1 g HD (three times a day)×10 days (adults), or pending order for Amoxicillin 400 mg/5 mL (weight-based) BID×10 days (pediatrics). Additionally, the choice of the medication such as the antibiotic is configurable.

For example, for patients with shoulder pain, the acts may include, but are not limited to, pending a referral to physical therapy; and 2) pending a shoulder X-ray.

For example, for patients with depression/anxiety, the acts may include, but are not limited to, 1) pending order for Sertraline 50 mg (1 tab daily, 30 tabs, 2 refills); 2) pending a referral to behavioral health/psychiatry. Additionally, the choice of antidepressant medication is configurable.

For example, for patients with sinusitis/nasal congestion/sinus pressure, the acts may include, but are not limited to, 1) pending order for Augmentin 875 mg/125 mg, 1 tab twice daily 7 days, 14 tabs, 0 refill; 2) pending order for Flonase (spray into each nostril twice daily, 1 unit, 3 refills). Additionally, the choice of the medication such as the antibiotic is configurable.

For example, for patients with vaginal discharge, the acts may include, but are not limited to, pending a NuSwab test.

For example, for patients with sexually transmitted disease (STD) exposure, the acts may include, but are not limited to, 1) pending a human immunodeficiency virus (HIV) test, a hepatitis panel test, a gonorrhea/chlamydia test, a Rapid Plasma Reagin (RPR) test, etc.

For example, for patients with abdominal pain, the acts may include, but are not limited to, pending a CMP test, a CBC test, a lipase test, a celiac panel test, an erythrocyte sedimentation rate (ESR) test, a Helicobacter pylori (H. pylori) breath test, etc.

For example, for patients with migraine headache, the acts may include, but are not limited to, 1) pending order for Sumatriptan 25 mg PO (by mouth)×1, may repeat dose after 2 h, 30 tabs, 1 refill; and 2) pending order for amitriptyline 10 mg PO qhs, 30 tabs, 1 refill. Additionally, and/or alternatively, the choice of the medication is configurable.

For example, for patients with gastroesophageal reflux disease (GERD)/acid reflux, the acts may include, but are not limited to pending order for omeprazole 20 mg qd (once a day), 30 capsules, 3 refills.

Use case 10 may be a case where a patient with multiple complaints makes a clinic visit.

At 10002, acts for use case 10 may be the same as the acts described with reference to use case 1 or use case 9.

The acts described herein are examples, and this disclosure is not limited thereto. The acts are configurable, upgradeable, and editable. Additional list of diagnoses may be added. Pended orders/tests are customizable by the care provider. The feature of pending orders/tests may be turned on/off by the care provider.

The use cases described herein are examples, and this disclosure is not limited thereto. The order in which the acts are described is not intended to be construed as a limitation, and any number of the described acts may be omitted or combined in any order and/or in parallel to implement the use cases.

In the above example use cases, the medical data associated with the patient may be acquired and arranged in an efficient way, facilitating the interaction between patients and medical care providers, decreasing the patient processing time in a clinical setting, streamlining the clinics' workflow, and/or improving the quality of care delivery.

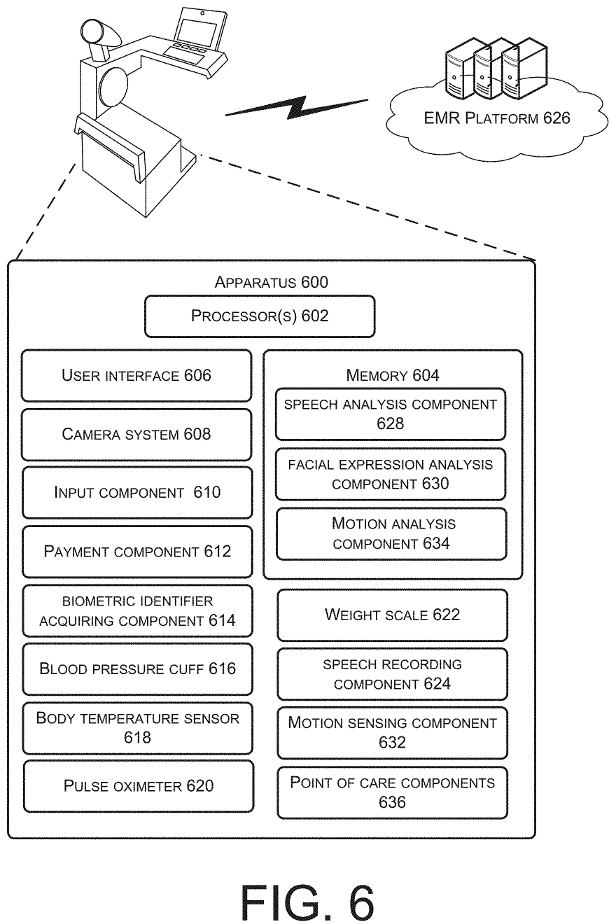

FIG. 6 illustrates an example schematic depiction of a system including apparatus 600 for decreasing patient processing time in a clinical setting according to implementations of this disclosure. In implementation, the apparatus 600 may include, but is not limited to, medical kiosks, check-in kiosks, interactive kiosks, self-service kiosks, smart kiosks, diagnostic kiosks, test kiosks, etc. In implementation, the apparatus 100 may be used in a clinical setting for the patient to conduct a self-check-in/rooming process.

In some examples, the apparatus 600 may be implemented similarly to the apparatus 100 described with respect to FIG. 1. Referring to FIG. 6, the apparatus 600 may include one or more processors 602, memory 604, a user interface 606, a camera system 608, an input component 610, a payment component 612, a biometric identifier acquiring component 614, and one or more physiological data acquiring components. In some examples, the one or more physiological data acquiring components may include, but are not limited to, a blood pressure cuff 616 configured to acquire a blood pressure associated with the patient, a body temperature sensor 618 configured to acquire a body temperature associated with the patient, a pulse oximeter 620 configured to acquire a blood oxygen saturation associated with the patient, and a weight scale 622 configured to acquire a weight associated with the patient.

The apparatus 600 may further include a speech recording component 624, configured to collect speech data from the patient. In some examples, the speech recording component 624 may be further configured to identify biomarkers rated to the tone, the rhythm, the resonance, the inflection, the tempo, the pacing, and the texture associated with the patient's voice. The apparatus 600 may communicate with the EMR platform 626 which keeps medical data associated with the patient and send the speech data associated with the patient to the EMR platform 626. The EMR platform 626 may update the medical record associated with the patient based on the speech data sent from the apparatus 600.

The memory 604 may further include a speech analysis component 628 which may be implemented as a machine learning model. Additionally, and/or alternatively, the speech analysis component 628 may be arranged at a remote computing system, an online server, a cloud server, the EMR platform 626, etc. The speech data may be fed to the speech analysis component 628 for further analysis. Any type of machine learning model may be used consistently with this disclosure. For example, machine learning algorithms may include, but are not limited to, regression algorithms (e.g., ordinary least squares regression (OLSR), linear regression, logistic regression, stepwise regression, multivariate adaptive regression splines (MARS), locally estimated scatterplot smoothing (LOESS)), instance-based algorithms (e.g., ridge regression, least absolute shrinkage and selection operator (LASSO), elastic net, least-angle regression (LARS)), decisions tree algorithms (e.g., classification and regression tree (CART), iterative dichotomiser 2 (ID2), Chi-squared automatic interaction detection (CHAID), decision stump, conditional decision trees), Bayesian algorithms (e.g., naïve Bayes, Gaussian naïve Bayes, multinomial naïve Bayes, average one-dependence estimators (AODE), Bayesian belief network (BNN), Bayesian networks), clustering algorithms (e.g., k-means, k-medians, expectation maximization (EM), hierarchical clustering), association rule learning algorithms (e.g., perceptron, back-propagation, hopfield network, Radial Basis Function Network (RBFN)), deep learning algorithms (e.g., Deep Boltzmann Machine (DBM), Deep Belief Networks (DBN), Convolutional Neural Network (CNN), Stacked Auto-Encoders), Dimensionality Reduction Algorithms (e.g., Principal Component Analysis (PCA), Principal Component Regression (PCR), Partial Least Squares Regression (PLSR), Sammon Mapping, Multidimensional Scaling (MDS), Projection Pursuit, Linear Discriminant Analysis (LDA), Mixture Discriminant Analysis (MDA), Quadratic Discriminant Analysis (QDA), Flexible Discriminant Analysis (FDA)), Ensemble Algorithms (e.g., Boosting, Bootstrapped Aggregation (Bagging), AdaBoost, Stacked Generalization (blending), Gradient Boosting Machines (GBM), Gradient Boosted Regression Trees (GBRT), Random Forest), SVM (support vector machine), supervised learning, unsupervised learning, semi-supervised learning, etc.

Additionally, other data associated with the patient may be input together with the speech data to the speech analysis component 628 for analysis, such as instantaneous physiological data (e.g., a blood pressure, a body temperature, a blood oxygen saturation, a weight, etc.), health history data (e.g., tobacco use data, alcohol use data, drug use data, surgery history data, etc.), social history data (e.g., family member health history data, childhood experience data, gender data, race data, ethnicity data, religion data, disability status data, etc.), data collected by other components of the apparatus 600, etc.

The speech analysis component 628 may be configured to output predictive analytics related to neurological and/or mental health conditions associated with the patient, including but not limited to cognitive impairment, dementia, speech disorders, depression, suicide, anxiety, bipolar disorder, schizophrenia, stroke, or the like. In some examples, the predictive analytics may indicate changes in health status and risk stratification of future adverse events associated with the patient. Additionally, the predictive analytics may be further used to generate recommendations of treatment and/or plans for the care provider (e.g., a physician, a certified nurse, a medical worker, etc.) based on specific risk(s) and conditions (s) identified.

The camera system 608 may be configured to collect facial expression data associated with the patient. In some examples, the camera system 608 may be further configured to identify biomarkers in relation to the patient's mood and/or mental health based on the facial expression data associated with the patient. The apparatus 600 may communicate with the EMR platform 626 which keeps medical data associated with the patient and send the facial expression data associated with the patient to the EMR platform 626. The EMR platform 626 may update the medical record associated with the patient based on the facial expression data sent from the apparatus 600.

The memory 604 may further include a facial expression analysis component 630 which may be implemented as a machine learning model. Additionally, and/or alternatively, the facial expression analysis component 630 may be arranged at a remote computing system, an online server, a cloud server, the EMR platform, etc. The facial expression data may be fed to the facial expression analysis component 630 for further analysis. Any type of machine learning model may be used consistently with this disclosure. Examples of machine learning algorithms are similar to those described above and are not repeated here.

Additionally, other data associated with the patient may be input together with the facial expression data to the facial expression analysis component 630 for analysis, such as instantaneous physiological data (e.g., a blood pressure, a body temperature, a blood oxygen saturation, a weight, etc.), health history data (e.g., tobacco use data, alcohol use data, drug use data, surgery history data, etc.), social history data (e.g., family member health history data, childhood experience data, gender data, race data, ethnicity data, religion data, disability status data, etc.), data collected by other components of the apparatus 600, or the like.

The facial expression analysis component 630 may be configured to output predictive analytics related to neurological and/or mental health conditions associated with the patient, including but not limited to Parkinson's disease, pain, depression, anxiety, suicidal ideation, drug addiction, substance abuse, malingering, or the like. In some examples, the predictive analytics may indicate changes in health status and risk stratification of future adverse events associated with the patient. Additionally, the predictive analytics may be further used to generate recommendations of treatment and/or plans for the care provider (e.g., a physician, a certified nurse, a medical worker, etc.) based on specific risk(s) and condition(s) identified.

The apparatus 600 may further include a motion sensing component 632 configured to collect motion data associated with the patient. In some examples, the motion sensing component 632 may be further configured to identify gait characteristics associated with the patient such as speed, balance, mobility, agility, range of motion, and the like, based on the motion data associated with the patient. The apparatus 600 may communicate with the EMR platform 626 which keeps medical data associated with the patient and send the motion data associated with the patient to the EMR platform 626. The EMR platform 626 may update the medical record associated with the patient based on the motion data sent from the apparatus 600.

The memory 604 may further include a motion analysis component 634 which may be implemented as a machine learning model. Additionally, and/or alternatively, the motion analysis component 634 may be arranged at a remote computing system, an online server, a cloud server, the EMR platform, etc. The motion data may be fed to the motion analysis component 634 for further analysis. Any type of machine learning model may be used consistently with this disclosure. Examples of machine learning algorithms are similar to those described above and are not repeated here.

Additionally, other data associated with the patient may be input together with the motion data to the motion analysis component 634 for analysis, such as instantaneous physiological data (e.g., a blood pressure, a body temperature, a blood oxygen saturation, a weight, etc.), health history data (e.g., tobacco use data, alcohol use data, drug use data, surgery history data, etc.), social history data (e.g., family member health history data, childhood experience data, gender data, race data, ethnicity data, religion data, disability status data, etc.), data collected by other components of the apparatus 600, or the like.

The motion analysis component 634 may be configured to output predictive analytics related to neurological and/or musculoskeletal and/or neurological disorders associated with the patient, including but not limited to, recurrent stroke, falls, progression of generalized weakness, Parkinson's disease, cognitive impairment, etc. In some examples, the predictive analytics may indicate changes in health status and risk stratification of future adverse events associated with the patient. Additionally, the predictive analytics may be further used to generate recommendations of treatment and/or plans for the care provider (e.g., a physician, a certified nurse, a medical worker, etc.) based on specific risk(s) and condition(s) identified. Additionally, the apparatus may provide one or more recommendations at least in part on the predictive analytics and the medical data associated with the patient. For example, if the motion data associated with the patient indicates that the patient has a shuffling and propulsive gait, the apparatus may provide the recommendation of getting self-help devices to meet daily needs and raising the toilet seat for the patient. As another example, if the motion data associated with the patient indicates that the patient has rigidity in the back, the apparatus may provide the recommendation of warm baths and massage to relax muscles.

The apparatus 600 may further include one or more Point of Care testing (POCT) components 636 configured to collect POCT data associated with the patient. The POCT components 636 may include, but are not limited to, a Covid/Flu testing component configured to collect Covid/Flu test data, a rapid strep testing component configured to collect streptococcus bacteria test data, a lipid testing component configured to collect lipid (e.g., cholesterol and triglycerides levels in the blood) test data, an HbA1C testing component configured to collect blood sugar level data, a complete blood count (CBC) component configured to blood cell count data, a Comprehensive Metabolic Panel (CMP) component configured to collect CMP data, a TSH (Thyroid-stimulating hormone) testing component configured to collect thyroid level data, etc. CMP is a blood test that gives doctors information about the patient's body fluid balance, levels of electrolytes like sodium and potassium, and how well the kidneys and liver are working.

The apparatus 600 may communicate with the EMR platform 626 which keeps medical data associated with the patient and send the POCT data associated with the patient to the EMR platform 626. The EMR platform 626 may update the medical record associated with the patient based on the facial expression data sent from the apparatus 600.

The apparatus 600 described herein may acquire and arrange the medical data associated with the patient in an efficient way, facilitating the interaction between patients and medical care providers, decreasing the patient processing time in a clinical setting, streamlining the clinics' workflow, and/or improving the quality of care delivery.

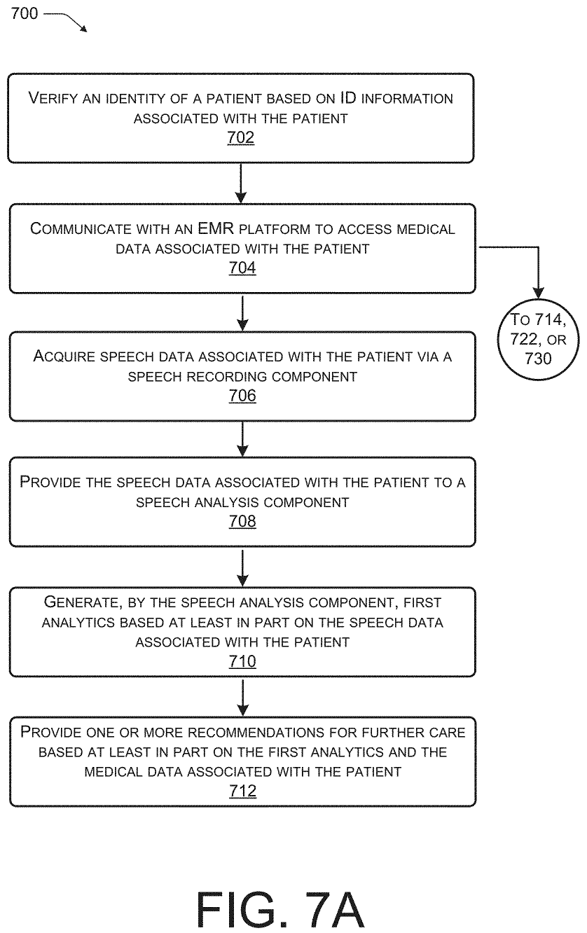

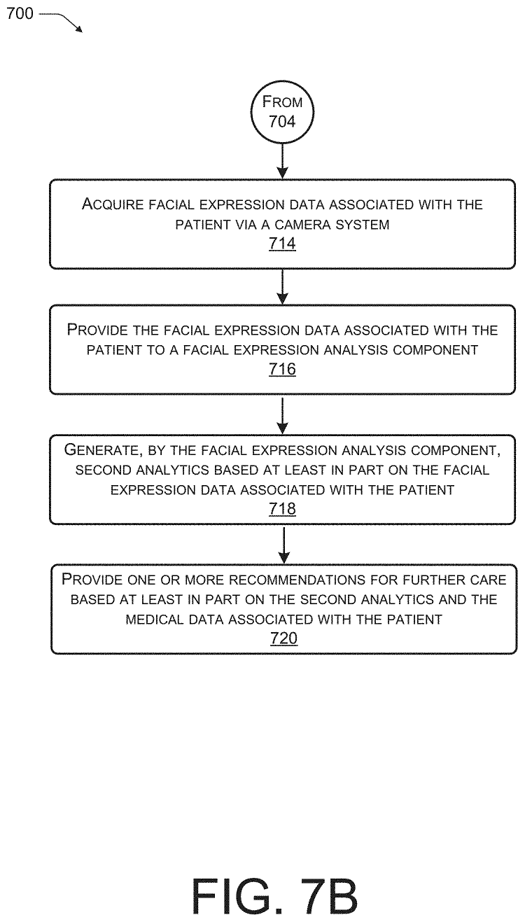

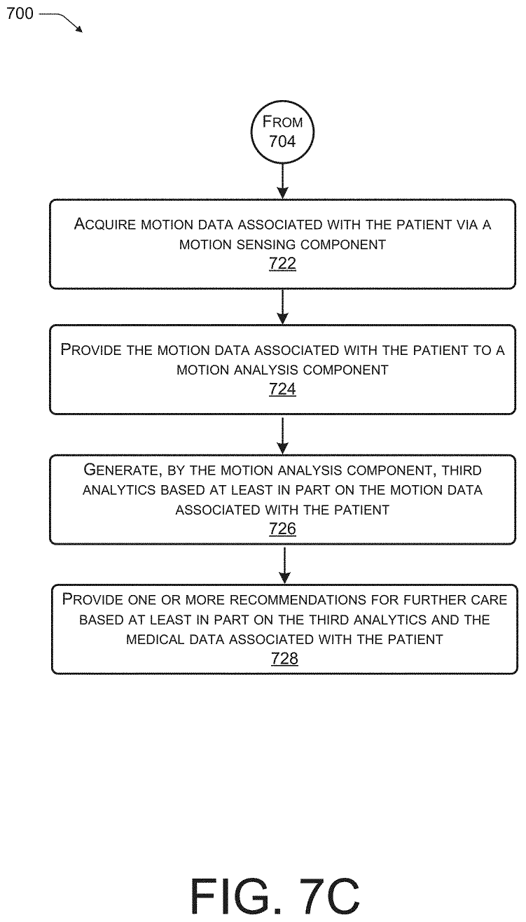

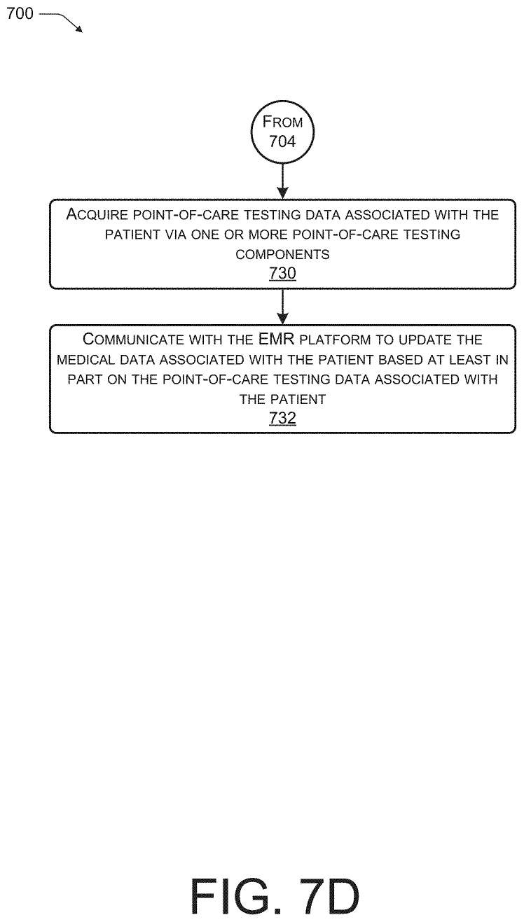

FIG. 7A, FIG. 7B, FIG. 7C, and FIG. 7D illustrate flow diagrams illustrating an example process for decreasing patient processing time in the clinical setting according to implementations of this disclosure.

Referring to FIG. 7A, process 700 may include the following operations. The process 700 is illustrated as flow graphs, each operation of which represents a sequence of operations that may be implemented in hardware, software, or a combination thereof. In the context of software, the operations represent computer-executable instructions stored on one or more computer-readable storage media that, when executed by one or more processors, perform the recited operations. Generally, computer-executable instructions include routines, programs, objects, components, data structures, and the like that perform particular functions or implement particular abstract data types. The order in which the operations are described is not intended to be construed as a limitation, and any number of the described operations may be omitted and/or combined in any order and/or in parallel to implement the processes. Some or all of the process 700 may be performed by systems and apparatus described throughout this disclosure, for example, the apparatus 100 described in FIG. 1, the apparatus 206 described in FIG. 2, the apparatus 600 described in FIG. 6, etc.

At 702, operations may include verifying an identity of a patient based on ID information associated with the patient. The ID information associated with the patient may include, but is not limited to, biometric identifiers, Email ID, etc. In implementation, the biometric identifier may include, but is not limited to, fingerprint, palm veins, face recognition, DNA, palm print, hand geometry, iris recognition, retina, odor, typing rhythm, gait, keystroke, signature, voice, etc.

At 704, operations may include communicating with an EMR platform to access medical data associated with the patient. The medical data associated with the patient may include, but is not limited to, the insurance information associated with the new patient, the demographic information associated with the new patient, the payment method, the medication list associated with the new patient, the medical/social history information associated with the new patient, etc. The medical/social history information may include, but is not limited to, tobacco use, alcohol use, drug use, etc.

At 706, operations may include acquiring speech data associated with the patient via a speech recording component. In implementation, an apparatus for decreasing patient processing time in the clinical setting (medical kiosks, check-in kiosks, interactive kiosks, self-service kiosks, smart kiosks, diagnostic kiosks, test kiosks, etc.) may present an instruction via a user interface, instructing the patient to read aloud a sentence (e.g., “you may′t teach an old dog new tricks,” etc.) displayed via the user interface. Then, the patient may read aloud a paragraph displayed via the user interface. The speech recording component of the apparatus may record the speech of the patient and store the speech data associated with the patient in storage in the apparatus.

At 708, operations may include providing the speech data associated with the patient to a speech analysis component. In implementation, the apparatus may include a speech analysis component configured to analyze the speech data associated with the patient. For example, the speech analysis component may be implemented as a machine learning model. Additionally, and/or alternatively, the speech analysis component may be arranged at a remote computing system, an online server, a cloud server, the EMR platform, etc. The speech data may be fed to the speech analysis component for further analysis. Any type of machine learning model may be used consistently with this disclosure. Examples of machine learning algorithms are similar to those described above and are not repeated here.

At 710, operations may include generating, by the speech analysis component, first analytics based at least in part on the speech data associated with the patient. In implementation, the speech analysis component may be configured to output first analytics related to neurological and/or mental health conditions associated with the patient, including but not limited to cognitive impairment, dementia, speech disorders, depression, suicide, anxiety, bipolar disorder, schizophrenia, stroke, etc. In some examples, the first analytics may indicate changes in health status and risk stratification of future adverse events associated with the patient.

At 712, operations may include providing one or more recommendations for further care based at least in part on the first analytics and the medical data associated with the patient. In implementation, the first analytics may be further used to generate recommendations of treatment and/or plans for the care provider (e.g., a physician, a certified nurse, a medical worker, etc.) based on specific risk(s) and condition(s) identified.

Referring to FIG. 7B, at 714, operations may include acquiring facial expression data associated with the patient via a camera system. In implementation, the apparatus may present an instruction via a user interface, instructing the patient to make a facial expression (e.g., “please smile,” “show your teeth,” etc.). Then, the camera system of the apparatus may record the facial expression of the patient and store the facial expression data associated with the patient in storage in the apparatus.

At 716, operations may include providing the facial expression data associated with the patient to a facial expression analysis component. In implementation, the apparatus may include a facial expression analysis component configured to analyze the facial expression data associated with the patient. For example, the facial expression analysis component may be implemented as a machine learning model. Additionally, and/or alternatively, the facial expression analysis component may be arranged at a remote computing system, an online server, a cloud server, the EMR platform, etc. The facial expression data may be fed to the facial expression analysis component for further analysis. Any type of machine learning model may be used consistently with this disclosure. Examples of machine learning algorithms are similar to those described above and are not repeated here.

At 718, operations may include generating, by the facial expression analysis component, second analytics based at least in part on the facial expression data associated with the patient. The facial expression analysis component may be configured to second predictive analytics related to neurological and/or mental health conditions associated with the patient, including but not limited to Parkinson's disease, pain, depression, anxiety, suicidal ideation, drug addiction, substance abuse, malingering, etc. In some examples, the second analytics may indicate changes in health status and risk stratification of future adverse events associated with the patient.

At 720, operations may include providing one or more recommendations for further care based at least in part on the second analytics and the medical data associated with the patient. In implementation, the second analytics may be further used to generate recommendations of treatment and/or plans for the care provider (e.g., a physician, a certified nurse, a medical worker, etc.) based on specific risk(s) and condition(s) identified.

Referring to FIG. 7C, at 722, operations may include acquiring motion data associated with the patient via a motion sensing component. In implementation, the apparatus may present an instruction via the user interface, instructing the patient to make moves (e.g., “close your eyes and hold both arms out for 10 seconds,” etc.). Then, the patient may follow the instruction to make the moves. A motion sensing component of the apparatus may record the motions of the patient and store the motion data associated with the patient in storage in the apparatus.

At 724, operations may include providing the motion data associated with the patient to a motion analysis component. In implementation, the apparatus may include a motion analysis component configured to analyze the motion data associated with the patient. For example, the motion analysis component may be implemented as a machine learning model. Additionally, and/or alternatively, the motion analysis component may be arranged at a remote computing system, an online server, a cloud server, the EMR platform, etc. The motion data may be fed to the motion analysis component for further analysis. Any type of machine learning model may be used consistently with this disclosure. Examples of machine learning algorithms are similar to those described above and are not repeated here.

At 726, operations may include generating, by the motion analysis component, third analytics based at least in part on the motion data associated with the patient. The motion analysis component may be configured to output predictive analytics related to neurological and/or musculoskeletal and/or neurological disorders associated with the patient, including but not limited to, recurrent stroke, falls, progression of generalized weakness, Parkinson's disease, cognitive impairment, etc. In some examples, the predictive analytics may indicate changes in health status and risk stratification of future adverse events associated with the patient.

At 728, operations may include providing one or more recommendations for further care based at least in part on the third analytics and the medical data associated with the patient. In implementation, the third analytics may be further used to generate recommendations of treatment and/or plans for the care provider (e.g., a physician, a certified nurse, a medical worker, etc.) based on specific risk(s) and condition(s) identified.

At 730, operations may include acquiring point-of-care testing data associated with the patient via one or more point-of-care testing components. The apparatus may further include one or more POCT components configured to collect POCT data associated with the patient. The POCT components may include, but are not limited to, a Covid/Flu testing component configured to collect Covid/Flu test data, a rapid strep testing component configured to collect streptococcus bacteria test data, a lipid testing component configured to collect lipid (e.g., cholesterol and triglycerides levels in the blood) test data, an HbA1C testing component configured to collect blood sugar level data, a complete blood count (CBC) component configured to blood cell count data, a Comprehensive Metabolic Panel (CMP) component configured to collect CMP data, a TSH (Thyroid-stimulating hormone) testing component configured to collect thyroid level data, etc. CMP is a blood test that gives doctors information about the patient's body fluid balance, levels of electrolytes like sodium and potassium, and how well the kidneys and liver are working.

At 732, operations may include communicating with the EMR platform to update the medical data associated with the patient based at least in part on the point-of-care testing data associated with the patient. The apparatus may communicate with the EMR platform which keeps medical data associated with the patient and send the POCT data associated with the patient to the EMR platform. The EMR platform may update the medical record associated with the patient based on the facial expression data sent from the apparatus.

The process 700 described herein may acquire and arrange the medical data associated with the patient in an efficient way, facilitating the interaction between patients and medical care providers, decreasing the patient processing time in a clinical setting, streamlining the clinics' workflow, and/or improving the quality of care delivery.

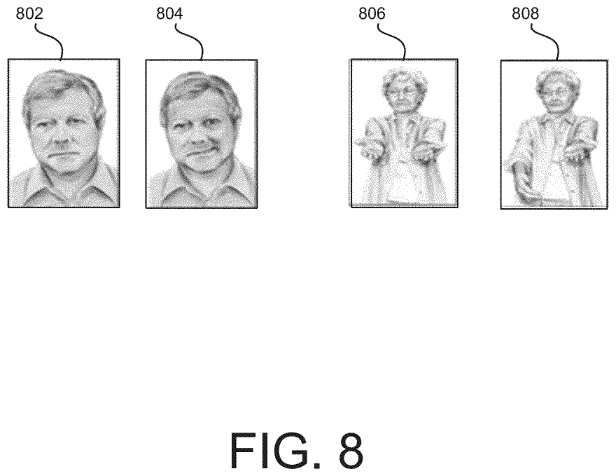

FIG. 8 illustrates examples of physiological traits associated with a patient that may be useful for generating predictive analytics associated with the patient.

Referring to FIG. 8, image 802 illustrates an example facial expression of a patient that may be deemed as normal, wherein both sides of the patient's face move equally when the patient is making a smile. Image 804 illustrates an example facial expression of a patient that may be deemed as abnormal, wherein one side of the patient's face does not move as well as the other side when the patient is making a smile.

In implementation, an apparatus for decreasing patient processing time in the clinical setting (medical kiosks, check-in kiosks, interactive kiosks, self-service kiosks, smart kiosks, diagnostic kiosks, test kiosks, etc.) may present an instruction via a user interface, instructing the patient to make a facial expression (e.g., “please smile,” “show your teeth,” etc.). Then, the camera system of the apparatus may record the facial expression of the patient and store the facial expression data associated with the patient in storage in the apparatus. The facial expression data may be fed to the facial expression analysis component of the apparatus for analysis. The facial expression analysis component of the apparatus may be configured to output predictive analytics associated with the patient based at least in part on the facial expression data associated with the patient. For example, if both sides of the patient's face move equally, the facial expression analysis component of the apparatus may determine that the patient's facial expression is normal. On the other hand, if one side of the patient's face does not move as well as the other side, the facial expression analysis component of the apparatus may determine that the patient's facial expression is abnormal.

Image 806 illustrates an example body motion of a patient that may be deemed as normal, wherein both arms of the patient move the same when the patient closes eyes and holds both arms out for several seconds (e.g., 5 seconds, 10 seconds, etc.). Image 808 illustrates an example body motion of a patient that may be deemed as abnormal, wherein one arm of the patient does not move, or one arm drifts down compared to the other arm.

In implementation, the apparatus may present an instruction via the user interface, instructing the patient to make moves (e.g., “close your eyes and hold both arms out for 10 seconds,” etc.). Then, the patient may follow the instruction to make the moves. A motion sensing component of the apparatus may record the motions of the patient and store the motion data associated with the patient in storage in the apparatus. The motion data may be fed to the motion analysis component of the apparatus for analysis. The motion analysis component of the apparatus may be configured to output predictive analytics associated with the patient based at least in part on the motion data associated with the patient. For example, if both arms of the patient move the same way, the motion analysis component of the apparatus may determine that the patient's move is normal. On the other hand, if one arm of the patient does not move, or one arm of the patient drifts down compared to the other arm, the motion analysis component of the apparatus may determine that the patient's move is abnormal.

Regarding speech analysis, the apparatus may present an instruction via a user interface, instructing the patient to read aloud a sentence displayed via the user interface. For example, “you can't teach an old dog new tricks.” Then, the patient may read aloud a sentence displayed via the user interface. The speech recording component of the apparatus may record the speech of the patient and store the speech data associated with the patient in storage in the apparatus. The speech data may be fed to the speech analysis component for analysis. The speech analysis component of the apparatus may be configured to output predictive analytics associated with the patient based at least in part on the speech data associated with the patient. For example, if the patient uses correct words with no slurring, the speech analysis component of the apparatus may determine that the speech of the patient is normal. On the other hand, if the patient slurs words, uses the wrong words, or is unable to speak, the speech analysis component of the apparatus may determine that the speech of the patient is abnormal.

In some examples, the speech data, the facial expression data, and the motion data associated with the patient may be used together for analysis. For example, if any one of the speech, facial expressions, and motions of the patient is abnormal, the machine learning model may determine that a likelihood of a stroke is 72%.

In implementations, the speech analysis component, the facial expression analysis component, and the motion analysis component may be implemented with machine learning model(s). A training dataset may be used to train the machine learning model(s). For example, the training dataset for training the machine learning model of the speech analysis component may include speech data and associated analytics (e.g., whether a patient's speech is normal or abnormal, whether the patient likely has cognitive impairment, dementia, speech disorders, depression, suicide, anxiety, bipolar disorder, schizophrenia, stroke, etc.). As another example, the training dataset for training the machine learning model of the facial expression analysis component may include facial expression data and associated analytics (e.g., whether a patient in an image is normal or abnormal, whether a patient in an image likely has Parkinson's disease, pain, depression, anxiety, suicidal ideation, drug addiction, substance abuse, malingering, etc.). As another example, the training dataset for training the machine learning model of the motion analysis component may include motion data and associated analytics (e.g., whether a patient in an image is normal or abnormal, whether a patient in an image likely has a recurrent stroke, falls, progression of generalized weakness, Parkinson disease, cognitive impairment, etc.). The training dataset is collected from various sources, for example, research centers, recovery centers, nursing homes, hospitals, clinics, emergency rooms, etc.

In some examples, the speech data, the facial expression data, and the motion data may be arranged in the same dataset to train machine learning model(s). Additionally, other data associated with the patient may be used together with speech data, facial expression data, and motion data to train the machine learning model(s), such as instantaneous physiological data (e.g., a blood pressure, a body temperature, a blood oxygen saturation, a weight, etc.), health history data (e.g., tobacco use data, alcohol use data, drug use data, surgery history data, etc.), social history data (e.g., family member health history data, childhood experience data, gender data, race data, ethnicity data, religion data, disability status data, etc.), etc. After training, the machine learning model(s) may produce predictive analytics based at least in part on the input data.

Some or all operations of the methods described above may be performed by execution of computer-readable instructions stored on a computer-readable storage medium, as defined below. The term “computer-readable instructions” as used in the description and claims, includes routines, applications, application modules, program modules, programs, components, data structures, algorithms, and the like. Computer-readable instructions may be implemented on various system configurations, including single-processor or multiprocessor systems, minicomputers, mainframe computers, personal computers, hand-held computing devices, microprocessor-based, programmable consumer electronics, combinations thereof, and the like.

The computer-readable storage media may include volatile memory (such as random access memory (RAM)) and/or non-volatile memory (such as read-only memory (ROM), flash memory, etc.). The computer-readable storage media may also include additional removable storage and/or non-removable storage including, but is not limited to, flash memory, magnetic storage, optical storage, and/or tape storage that may provide non-volatile storage of computer-readable instructions, data structures, program modules, and the like.

A non-transient computer-readable storage medium is an example of computer-readable media. Computer-readable media includes at least two types of computer-readable media, namely computer-readable storage media and communications media. Computer-readable storage media includes volatile and non-volatile, removable and non-removable media implemented in any process or technology for storage of information such as computer-readable instructions, data structures, program modules, or other data. Computer-readable storage media includes, but is not limited to, phase-change memory (PRAM), static random-access memory (SRAM), dynamic random-access memory (DRAM), other types of random-access memory (RAM), read-only memory (ROM), electrically erasable programmable read-only memory (EEPROM), flash memory or other memory technology, compact disc read-only memory (CD-ROM), digital versatile discs (DVD) or other optical storage, magnetic cassettes, magnetic tape, magnetic disk storage or other magnetic storage devices, or any other non-transmission medium that may be used to store information for access by a computing device. In contrast, communication media may embody computer-readable instructions, data structures, program modules, or other data in a modulated data signal, such as a carrier wave, or other transmission mechanisms. As defined herein, computer-readable storage media do not include communication media.

The computer-readable instructions stored on one or more non-transitory computer-readable storage media that, when executed by one or more processors, may perform operations described above with reference to the drawings. Generally, computer-readable instructions include routines, programs, objects, components, data structures, and the like that perform particular functions or implement particular abstract data types. The order in which the operations are described is not intended to be construed as a limitation, and any number of the described operations may be omitted or combined in any order and/or in parallel to implement the processes.

### CONCLUSION

Although the subject matter has been described in language specific to structural features and/or methodological acts, it is to be understood that the subject matter defined in the appended claims is not necessarily limited to the specific features or acts described. Rather, the specific features and acts are disclosed as illustrative forms of implementing the claims.

### EXAMPLE CLAUSES

Clause 1. A method for decreasing patient processing time in a clinical setting, the method comprising: verifying an identity of a patient based on identification (ID) information associated with the patient; communicating with an Electronic Medical Record (EMR) platform to: access previously entered medical data associated with the patient, when the patient is a returning patient; or create a new account associated with the patient, when the patient is a new patient; presenting, via an interface of an apparatus, instructions for acquiring instantaneous physiological data associated with the patient; acquiring the instantaneous physiological data associated with the patient via one or more physiological data acquiring components of the apparatus; and communicating with the EMR platform to update the medical data associated with the patient based on the instantaneous physiological data associated with the patient, and provide one or more recommendations for further care based on the medical data associated with the patient.

Clause 2. The method of clause 1, wherein the instantaneous physiological data includes at least one of systolic blood pressure, diastolic blood pressure, body temperature, weight, heart rate, pulse rate, respiratory rate, or pulse oximetry.

Clause 3. The method of clause 1, wherein the verifying the identity of the patient based on the ID information associated with the patient includes: requesting, via the interface of the apparatus, the ID information associated with the patient; receiving, via the apparatus, the ID information associated with the patient; and determining, based on the ID information associated with the patient, the identity associated with the patient to correlate the identity with the medical data associated with the patient.

Clause 4. The method of clause 1, further comprising: presenting a verification request of current validity of previously entered medical data associated with the patient, when the previously entered medical data associated with the patient is available; and providing an edit option for previously entered medical data associated with the patient.

Clause 5. The method of clause 1, further comprising: presenting a request for a reason for a visit; receiving the reason for the visit associated with the patient, wherein a standard template corresponding to the reason for the visit associated with the patient is provided; and communicating with the EMR platform to update the medical data associated with the patient based on the reason for the visit associated with the patient and the standard template corresponding to the reason for the visit associated with the patient.

Clause 6. The method of clause 1, wherein the one or more recommendations for further care includes at least one of providing advice and/or recommendations about a medical condition, diagnosing and/or treating a medical condition, providing referral services for a medical condition, prescribing medicine for a medical condition, periodically monitoring a medical condition, providing follow-up checks for a medical condition, providing routine check-up services, providing advice, counseling, and/or recommendations about medical and/or health matters, providing a course of treatment for a medical condition, providing health counseling, providing health information, providing wellness counseling, and/or providing wellness information.

Clause 7. The method of clause 1, wherein the medical data associated with the patient and/or the instantaneous physiological data associated with the patient are converted into a graph over a time interval.

Clause 8. The method of clause 1, further comprising communicating, by a terminal device associated with a care provider, with the EMR platform to update the medical data associated with the patient based on a confirmation of a respective recommendation of the one or more recommendations for further care.

Clause 9. The method of clause 1, further comprising communicating, by a terminal device associated with a care provider, with the EMR platform to update the medical data associated with the patient based on a denial of a respective recommendation of the one or more recommendations for further care.

Clause 10. The method of clause 1, further comprising communicating, by a terminal device associated with a care provider, with the EMR platform to update the medical data associated with the patient based on a correction of a respective recommendation of the one or more recommendations for further care.

Clause 11. The method of clause 1, further comprising communicating, by a terminal device associated with a care provider, with the EMR platform to update the medical data associated with the patient based on diagnostic information associated with the patient.

Clause 12. A system for decreasing patient processing time in a clinical setting, the system comprising: an apparatus in the clinical setting, including: one or more processors, memory, coupled to the one or more processors, the memory storing thereon computer-readable instructions executable by the one or more processors, an interface configured to present and/or receive information, and one or more physiological data acquiring components; and a terminal device associated with a care provider; wherein the computer-readable instructions stored on the memory, when executed by the one or more processors, cause the one or more processors to perform acts including: verifying an identity of a patient based on identification (ID) information associated with the patient; communicating with an Electronic Medical Record (EMR) platform to: access previously entered medical data associated with the patient, when the patient is a returning patient; or create a new account associated with the patient, when the patient is a new patient; presenting, via an interface of an apparatus, instructions for acquiring instantaneous physiological data associated with the patient; acquiring the instantaneous physiological data associated with the patient via one or more physiological data acquiring components of the apparatus; and communicating, by the apparatus, with the EMR platform to update the medical data associated with the patient based on the instantaneous physiological data associated with the patient, and by providing one or more recommendations for further care based on the medical data associated with the patient.

Clause 13. The system of clause 12, wherein the apparatus in the clinical setting further includes one or more point-of-care testing components, the one or more point-of-care testing components including an ultrasound scan component, a urine dipstick test component, an electrocardiogram (EKG) sensor, a rapid strep test component, a flu test component, a glucose check component, an Hemoglobin A1c (HbA1c) check component, a thyroid-stimulating hormone (TSH) test component, a lipid test component, a coronavirus disease (COVID-19) test component, an edema test component, an ultrasound exam component, and/or an eye exam component.

Clause 14. The system of clause 12, wherein the terminal device associated with the care provider is configured to communicate with the EMR platform to update the medical data associated with the patient based on a confirmation, a denial, and/or a correction of a respective recommendation of the one or more recommendations.

Clause 15. The system of clause 12, wherein the terminal device associated with the care provider is configured to communicate with the EMR platform to update the medical data associated with the patient based on diagnostic information associated with the patient.

Clause 16. An apparatus for decreasing a patient processing time in a clinical setting, the apparatus comprising: one or more processors; memory, coupled to the one or more processors, the memory storing thereon computer-readable instructions executable by the one or more processors; an interface, coupled to the one or more processors, and configured to present and/or receive information; and one or more physiological data acquiring components, coupled to the one or more processors, and configured to acquire instantaneous physiological data associated with the patient; wherein the computer-readable instructions stored on the memory, when executed by the one or more processors, cause the apparatus to perform acts including: verifying an identity of a patient based on identification (ID) information associated with the patient; communicating with an Electronic Medical Record (EMR) platform to: access previously entered medical data associated with the patient, when the patient is a returning patient; or create a new account associated with the patient, when the patient is a new patient; presenting, via an interface of an apparatus, instructions for acquiring instantaneous physiological data associated with the patient; acquiring the instantaneous physiological data associated with the patient via one or more physiological data acquiring components of the apparatus; and communicating, by the apparatus, with the EMR platform to update the medical data associated with the patient based on the instantaneous physiological data associated with the patient, and by providing one or more recommendations for further care based on the medical data associated with the patient.

Clause 17. The apparatus of clause 16, wherein the instantaneous physiological data includes at least one of systolic blood pressure, diastolic blood pressure, body temperature, weight, heart rate, pulse rate, respiratory rate, or pulse oximetry.

Clause 18. The apparatus of clause 16, the acts further comprising: presenting a request for a reason for a visit; receiving the reason for the visit associated with the patient, wherein a standard template corresponding to the reason for the visit associated with the patient is provided; and communicating with the EMR platform to update the medical data associated with the patient based on the reason for the visit associated with the patient and the standard template corresponding to the reason for the visit associated with the patient.

Clause 19. The apparatus of clause 16, further comprising one or more point-of-care testing components, the one or more point-of-care testing components including an ultrasound scan component, a urine dipstick test component, an electrocardiogram (EKG) sensor, a rapid strep test component, a flu test component, a glucose check component, an Hemoglobin A1c (HbA1c) check component, a thyroid-stimulating hormone (TSH) test component, a lipid test component, a coronavirus disease (COVID-19) test component, an edema test component, an ultrasound exam component, and/or an eye exam component.

Clause 20. The apparatus of clause 16, wherein the one or more processors are configured to execute an Artificial Intelligence (AI) mechanism.

Clause 21. A computer-readable storage medium storing computer-readable instructions executable by one or more processors, that when executed by the one or more processors, causes the one or more processors to perform acts comprising: verifying an identity of a patient based on identification (ID) information associated with the patient; communicating with an Electronic Medical Record (EMR) platform to: access previously entered medical data associated with the patient, when the patient is a returning patient; or create a new account associated with the patient, when the patient is a new patient; presenting, via an interface of an apparatus, instructions for acquiring instantaneous physiological data associated with the patient; acquiring the instantaneous physiological data associated with the patient via one or more physiological data acquiring components of the apparatus; and communicating, by the apparatus, with the EMR platform to update the medical data associated with the patient based on the instantaneous physiological data associated with the patient, and by providing one or more recommendations for further care based on the medical data associated with the patient.

Clause 22: A system for decreasing patient processing time in a clinical setting, the system comprising: one or more processors; and memory, coupled to the one or more processors, the memory storing thereon computer-readable instructions executable by the one or more processors, when executed by the one or more processors, cause the one or more processors to perform acts including: verifying an identity of a patient based on identification (ID) information associated with the patient, communicating with an Electronic Medical Record (EMR) platform to access medical data associated with the patient, acquiring speech data associated with the patient via a speech recording component, providing the speech data associated with the patient to a speech analysis component, generating, by the speech analysis component, first analytics based at least in part on the speech data associated with the patient, and communicating with the EMR platform to update the medical data associated with the patient based at least in part on the speech data associated with the patient.

Clause 23: The system of paragraph 22, the acts further including providing one or more recommendations for further care based at least in part on the first analytics and the medical data associated with the patient.

Clause 24: The system of either paragraph 22 or 23, wherein the speech analysis component includes a first machine learning model.

Clause 25: The system of any one of paragraphs 22-24, the acts further including: acquiring facial expression data associated with the patient via a camera system; providing the facial expression data associated with the patient to a facial expression analysis component; generating, by the facial expression analysis component, second analytics based at least in part on the facial expression data associated with the patient; and communicating with the EMR platform to update the medical data associated with the patient based at least in part on the facial expression data associated with the patient.

Clause 26: The system of paragraph 25, the acts further including providing one or more recommendations for further care based at least in part on the second analytics and the medical data associated with the patient.

Clause 27: The system of either paragraph 25 or 26, wherein the speech analysis component includes a second machine learning model.

Clause 28: The system of any one of paragraphs 22-27, the acts further including: acquiring motion data associated with the patient via a motion sensing component; providing the motion data associated with the patient to a motion analysis component; generating, by the motion analysis component, third analytics based at least in part on the motion data associated with the patient; and communicating with the EMR platform to update the medical data associated with the patient based at least in part on the motion data associated with the patient.

Clause 29: The system of paragraph 28, the acts further comprising providing one or more recommendations for further care based at least in part on the third analytics and the medical data associated with the patient.

Clause 30: The system of either paragraph 28 or 29, wherein the motion analysis component includes a third machine learning model.

Clause 31: The system of any one of paragraphs 22-30, further comprising one or more point-of-care testing components, the one or more point-of-care testing components including a rapid strep test component, a coronavirus disease (COVID-40) test component, a flu test component, a Hemoglobin A1c (HbA1c) check component, a lipid test component, a complete blood count (CBC) component, a Comprehensive Metabolic Panel (CMP) component, and/or a thyroid-stimulating hormone (TSH) test component.

Clause 32: A method for decreasing patient processing time in a clinical setting, the method comprising: verifying an identity of a patient based on identification (ID) information associated with the patient; communicating with an Electronic Medical Record (EMR) platform to access medical data associated with the patient; acquiring speech data associated with the patient via a speech recording component; providing the speech data associated with the patient to a speech analysis component; generating, by the speech analysis component, first analytics based at least in part on the speech data associated with the patient; and communicating with the EMR platform to update the medical data associated with the patient based on the speech data associated with the patient.

Clause 33: The method of paragraph 32, further comprising providing one or more recommendations for further care based at least in part on the first analytics and the medical data associated with the patient.

Clause 34: The method of either paragraph 32 or 33, further comprising: acquiring facial expression data associated with the patient via a camera system; providing the facial expression data associated with the patient to a facial expression analysis component; generating, by the facial expression analysis component, second analytics based at least in part on the facial expression data associated with the patient; and communicating with the EMR platform to update the medical data associated with the patient based at least in part on the facial expression data associated with the patient.

Clause 35: The method of paragraph 34, further comprising providing one or more recommendations for further care based at least in part on the second analytics and the medical data associated with the patient.

Clause 36: The method of any one of paragraphs 32-35, further comprising: acquiring motion data associated with the patient via a motion sensing component; providing the motion data associated with the patient to a motion analysis component; generating, by the motion analysis component, third analytics based at least in part on the motion data associated with the patient; and communicating with the EMR platform to update the medical data associated with the patient based at least in part on the motion data associated with the patient.

Clause 37: The method of paragraph 36, further comprising providing one or more recommendations for further care based at least in part on the third analytics and the medical data associated with the patient.

Clause 38: The method of any one of paragraphs 32-37, further comprising: acquiring point-of-care testing data associated with the patient via one or more point-of-care testing components; and communicating with the EMR platform to update the medical data associated with the patient based at least in part on the point-of-care testing data associated with the patient.

Clause 39: The method of paragraph 38, wherein the point-of-care testing data includes at least one of: rapid strep test data, coronavirus disease (COVID-40) test data, flu test data, Hemoglobin A1c (HbA1c) check data, lipid test data, complete blood count (CBC) data, comprehensive metabolic panel (CMP) data, or thyroid-stimulating hormone (TSH) test data.

Clause 40: A computer-readable storage medium storing computer-readable instructions executable by one or more processors, that when executed by the one or more processors, causes the one or more processors to perform acts comprising: verifying an identity of a patient based on identification (ID) information associated with the patient; communicating with an Electronic Medical Record (EMR) platform to access medical data associated with the patient; acquiring speech data associated with the patient via a speech recording component; providing the speech data associated with the patient to a speech analysis component; generating, by the speech analysis component, first analytics based at least in part on the speech data associated with the patient; and communicating with the EMR platform to update the medical data associated with the patient based on the speech data associated with the patient.

Clause 41: The computer-readable storage medium of paragraph 40, the acts further comprising providing one or more recommendations for further care based at least in part on the first analytics and the medical data associated with the patient.

While the example clauses described above are described with respect to one particular implementation, it should be understood that, in the context of this document, the content of the example clauses may also be implemented via a method, device, system, computer-readable medium, and/or another implementation. Additionally, any of clauses 1-41 may be implemented alone or in combination with any other one or more of the clauses 1-41.

What is claimed is:

1. A system for decreasing patient processing time in a clinical setting, the system comprising:
one or more processors; and
memory, coupled to the one or more processors, the memory storing thereon computer-readable instructions executable by the one or more processors, when executed by the one or more processors, cause the one or more processors to perform acts including:
verifying an identity of a patient based on identification (ID) information associated with the patient,
communicating with an Electronic Medical Record (EMR) platform to access medical data associated with the patient,
acquiring speech data associated with the patient via a speech recording component,
providing the speech data associated with the patient to a speech analysis component,
generating, by the speech analysis component, first analytics based at least in part on the speech data associated with the patient, and
communicating with the EMR platform to update the medical data associated with the patient based at least in part on the speech data associated with the patient.

2. The system of claim 1, the acts further including providing one or more recommendations for further care based at least in part on the first analytics and the medical data associated with the patient.

3. The system of claim 1, wherein the speech analysis component includes a first machine learning model.

4. The system of claim 1, the acts further including:
acquiring facial expression data associated with the patient via a camera system;
providing the facial expression data associated with the patient to a facial expression analysis component;
generating, by the facial expression analysis component, second analytics based at least in part on the facial expression data associated with the patient; and
communicating with the EMR platform to update the medical data associated with the patient based at least in part on the facial expression data associated with the patient.

5. The system of claim 4, the acts further including providing one or more recommendations for further care based at least in part on the second analytics and the medical data associated with the patient.

6. The system of claim 4, wherein the speech analysis component includes a second machine learning model.

7. The system of claim 1, the acts further including:
acquiring motion data associated with the patient via a motion sensing component;
providing the motion data associated with the patient to a motion analysis component;
generating, by the motion analysis component, third analytics based at least in part on the motion data associated with the patient; and
communicating with the EMR platform to update the medical data associated with the patient based at least in part on the motion data associated with the patient.

8. The system of claim 7, the acts further comprising providing one or more recommendations for further care based at least in part on the third analytics and the medical data associated with the patient.

9. The system of claim 7, wherein the motion analysis component includes a third machine learning model.

10. The system of claim 1, further comprising one or more point-of-care testing components, the one or more point-of-care testing components including a rapid strep test component, a coronavirus disease (COVID-19) test component, a flu test component, a Hemoglobin A1c (HbA1c) check component, a lipid test component, a complete blood count (CBC) component, a Comprehensive Metabolic Panel (CMP) component, and/or a thyroid-stimulating hormone (TSH) test component.

11. A method for decreasing patient processing time in a clinical setting, the method comprising:
verifying an identity of a patient based on identification (ID) information associated with the patient;
communicating with an Electronic Medical Record (EMR) platform to access medical data associated with the patient;
acquiring speech data associated with the patient via a speech recording component;
providing the speech data associated with the patient to a speech analysis component;
generating, by the speech analysis component, first analytics based at least in part on the speech data associated with the patient; and
communicating with the EMR platform to update the medical data associated with the patient based on the speech data associated with the patient.

12. The method of claim 11, further comprising providing one or more recommendations for further care based at least in part on the first analytics and the medical data associated with the patient.

13. The method of claim 11, further comprising:
acquiring facial expression data associated with the patient via a camera system;
providing the facial expression data associated with the patient to a facial expression analysis component;
generating, by the facial expression analysis component, second analytics based at least in part on the facial expression data associated with the patient; and
communicating with the EMR platform to update the medical data associated with the patient based at least in part on the facial expression data associated with the patient.

14. The method of claim 13, further comprising providing one or more recommendations for further care based at least in part on the second analytics and the medical data associated with the patient.

15. The method of claim 11, further comprising:
acquiring motion data associated with the patient via a motion sensing component;
providing the motion data associated with the patient to a motion analysis component;
generating, by the motion analysis component, third analytics based at least in part on the motion data associated with the patient; and
communicating with the EMR platform to update the medical data associated with the patient based at least in part on the motion data associated with the patient.

16. The method of claim 15, further comprising providing one or more recommendations for further care based at least in part on the third analytics and the medical data associated with the patient.

17. The method of claim 11, further comprising:
acquiring point-of-care testing data associated with the patient via one or more point-of-care testing components; and
communicating with the EMR platform to update the medical data associated with the patient based at least in part on the point-of-care testing data associated with the patient.

18. The method of claim 17, wherein the point-of-care testing data includes at least one of:
rapid strep test data,
coronavirus disease (COVID-19) test data,
flu test data,
Hemoglobin A1c (HbA1c) check data,
lipid test data,
complete blood count (CBC) data,
comprehensive metabolic panel (CMP) data, or
thyroid-stimulating hormone (TSH) test data.

19. A computer-readable storage medium storing computer-readable instructions executable by one or more processors, that when executed by the one or more processors, causes the one or more processors to perform acts comprising:
verifying an identity of a patient based on identification (ID) information associated with the patient;
communicating with an Electronic Medical Record (EMR) platform to access medical data associated with the patient;
acquiring speech data associated with the patient via a speech recording component;
providing the speech data associated with the patient to a speech analysis component;
generating, by the speech analysis component, first analytics based at least in part on the speech data associated with the patient; and
communicating with the EMR platform to update the medical data associated with the patient based on the speech data associated with the patient.

20. The computer-readable storage medium of claim 19, the acts further comprising providing one or more recommendations for further care based at least in part on the first analytics and the medical data associated with the patient.
